(19) United States
(12) Patent Application Publication
(10) Pub. No.: US 2019/0350964 A1
(43) Pub. Date: Nov. 21, 2019

(54) ANTI-CANCER COMPOUNDS AND USES THEREOF

(71) Applicant: NATIONAL UNIVERSITY OF SINGAPORE, Singapore (SG)

(72) Inventors: Sang Hyun LEE, Singapore (SG); Mayumi KITAGAWA, Singapore (SG); David Michael EPSTEIN, Singapore (SG)

(21) Appl. No.: 16/467,937

(22) PCT Filed: Dec. 8, 2017
(86) PCT No.: PCT/SG2017/050608

§ 371 Date: Jun. 7, 2019

(30) Foreign Application Priority Data
Dec. 8, 2016 (SG) 10201610300X

(51) Int. Cl. A61K 31/7105; A61K 31/435; A61P 35/00; C12Q 1/48; G01N 33/574; G01N 33/50

(52) U.S. Cl. CPC A61K 31/7105; A61K 31/435; A61P 35/00; G01N 2500/10; G01N 33/57484; G01N 33/5011; G01N 2333/91215; C12Q 1/485

(57) ABSTRACT

Compounds and their use in modulating the Ras/Raf/MEK/ERK and PI3K/Akt/mTOR signaling pathways to protect normal cells in scenarios such as chemotherapy to kill cancer cells are provided. The compounds inhibit phosphatidylinositol 5-phosphate 4-kinase (PI5P4K) and/or increase phosphoinositide 3-kinase-interacting protein 1 (PIK31P1). Also provided are methods for identifying such compounds, methods of treatment using same and other uses.

### FIELD OF THE INVENTION

The present invention relates to compounds and their use in modulating the Ras/Raf/MEK/ERK and PI3K/Akt/mTOR signaling pathways to protect normal cells in scenarios such as chemotherapy to kill cancer cells. More particularly, the compounds modulate phosphatidylinositol 5-phosphate 4-kinase (PI5P4K) and/or phosphoinositide 3-kinase-interacting protein 1 (PIK3IP1). Also provided are methods for identifying such compounds, methods of treatment using same and other uses.

### BACKGROUND OF THE INVENTION

Achieving robust cancer-specific tumor cell lethality is the ultimate clinical goal. The Ras/Raf/MEK/ERK and PI3K/Akt/mTOR signaling pathways are essential for cell survival and proliferation in response to external cues. Mutation of proteins within these pathways are among the most common oncogenic targets in human cancers (McCormick, F. Clin. Cancer Res. 21: 1797-1801 (2015); Mayer, I. A. & Arteaga, C. L. Annu. Rev. Med. 67: 11-28 (2016)), and this has spawned a longstanding effort to develop selective inhibitors of these pathways for cancer therapy. Unfortunately, there is ample evidence that cross-talk or cross-amplification of signaling events occurs between these pathways, which both positively and negatively regulate downstream cellular growth events. Moreover, the antitumor activities of single-agent targeted therapies directed to block these signaling pathways has generally been disappointing with an unintended pathway activation leading to drug resistance. This has prompted the testing of multiple targeted therapies in combination in order to inhibit multiple oncogenic dependencies. However, combined treatment with drugs that target the Ras/Raf/MEK/ERK and PI3K/Akt/mTOR signaling pathways has met with marginal clinical success (Jokinen, E. & Koivunen, J. P. Ther. Adv. Med. Oncol. 7: 170-180 (2015)). Thus, there remains the ultimate goal of identifying targets that mediate resistance and cross-talk between these two central pathways.

### SUMMARY OF THE INVENTION

According to a first aspect, a preferred embodiment of the present invention provides an in vitro or in vivo method for modulating cell survival, comprising contacting at least one cell with at least one phosphatidylinositol 5-phosphate 4-kinase family (PI5P4Ks) modulator and/or at least one phosphoinositide 3-kinase-interacting protein 1 (PIK3IP1) modulator.

According to another aspect, the present invention provides a method for identifying compounds that modulate PI5P4Ks activity and are suitable for use in treating a hyperproliferative disorder or disease, said method comprising:

(a) providing at least one cell comprising said PI5P4Ks;
(b) contacting said at least one cell with at least one test compound;
(c) detecting whether PI5P4Kα, PI5P4Kβ and/or PI5P4Kγ activity is inhibited and the at least one cell enters cell cycle arrest at G1/S phase, and comparing with untreated cells.

According to another aspect, a preferred embodiment of the present invention provides a method of treatment of a hyperproliferative disease or disorder, which method comprises the administration of an effective amount of at least one compound which inhibits PI5P4K activity and causes normal cells to enter cell cycle arrest at G1/S phase but has no effect on transformed or hyperproliferating cells, and an effective amount of an anti-hyperproliferation agent.

In a preferred embodiment, the at least one compound inhibits PI5P4Kα, PI5P4Kβ and/or PI5P4Kγ activity and causes normal cells to enter cell cycle arrest at G1/S phase but has no effect on transformed or hyperproliferating cells.

According to another aspect, a preferred embodiment of the present invention provides a method of treatment of a hyperproliferative disease or disorder, which method comprises the administration of an effective amount of a compound which inhibits PI5P4Kα, PI5P4Kβ and/or PI5P4Kγ activity, thereby causing normal cells to enter cell cycle arrest at G1/S phase, and wherein said compound also causes cells of said hyperproliferative disease to undergo mitotic catastrophe.

According to another aspect, a preferred embodiment of the present invention provides use of at least one modulator that inhibits PI5P4Kα, PI5P4Kβ and/or PI5P4Kγ activity and causes normal cells to enter cell cycle arrest at G1/S phase while having no effect on transformed or hyperproliferating cells, for the preparation of a medicament for the treatment of a hyperproliferative disease or disorder in combination with an antiproliferative agent.

According to another aspect, a preferred embodiment of the present invention provides use of at least one modulator that inhibits PI5P4Kα, PI5P4Kβ and/or PI5P4Kγ activity and causes normal cells to enter cell cycle arrest at G1/S phase and wherein, additionally, said compound also causes transformed or hyperproliferating cells to undergo mitotic catastrophe for the preparation of a medicament for the treatment of a hyperproliferative disease or disorder.

### BRIEF DESCRIPTION OF THE FIGURES

FIGS. 1A-1K show selective killing effects of compound a131 in cancer cells. FIG. 1A: Structure of a131. FIG. 1B: Crystal violet assay of isogenic normal and transformed BJ cells treated with a131 for 72 h. FIGS. 1C-1D: Normal and transformed BJ cells were treated with a131 at 2.5 μM for 48 h. FACS analysis using BrdU and PI double staining of the indicated cells. Percentages of BrdU positive (S) and subG1 (<2N) population are shown (FIG. 1C). Immunoblot analysis of cleaved PARP and caspase-3 (Cas-3) (FIG. 1D). FIG. 1E: MTT assay of human normal and cancer cell lines treated with a131 for 72 h (n=3). Mean concentration values for a131 to achieve 50% growth inhibition (GI₅₀) in each cell line are plotted with ±S.D. FIG. 1F: FACS analysis of a series of engineered BJ-derived fibroblasts treated with a131 at 5 μM for 48 h. Mean values of subG1 (<2N) population ±S.D. are shown (n>3). FIGS. 1G-1H: Normal and transformed BJ cells stably expressing GFP-histone H2B were treated with a131 at 2.5 μM for 32 h prior to fixation. Immunofluorescence analysis with representative images (FIG. 1G). Quantification of cells (n>100 per condition) with the indicated centrosome numbers. Mean values±S.D. are shown (n=3) (FIG. 1H). FIG. 1I: Mice bearing established tumor xenografts were treated orally (PO) or intraperitoneally (IP) twice daily with the indicated doses of a131 or its derivative b5. Tumor volumes were calculated periodically as indicated. Paclitaxel (PTX) was administered intravenously for twice every 4 days. Mean tumor volumes±S.D. from 6 mice are shown. Two-way ANOVA was performed to determine statistical significance compared to vehicle control. FIGS. 1J-1K: TUNEL staining (FIG. 1J) or immunohistochemical analysis (FIG. 1K) of HCT-15 tumor sections on Day 12 with representative images (top). FIG. 1J: The percentage of TUNEL-positive cells were calculated from tumor sections (bottom, 5 cropped images of more than 6 sections from each group). FIG. 1K: Mean values of cell populations (n>100 per condition) with multipolar mitotic-spindles ±S.D. are shown (bottom). White bars, 5 μm. Where indicated, two-tailed unpaired t tests were performed to determine statistical significance.

FIGS. 2A-2E show the selective killing effects of compound a131 in transformed BJ cells without inducing genotoxic stress. FIG. 2A: Normal and transformed BJ cells were treated with a131 at a range of different concentrations (from 0.1 μM to 40 μM) for 72 h in triplicate and cell viability was determined by MTT assay in comparison with indicated antiproliferative agents. Mean values with ±standard deviation (S.D.) are shown (n=3). FIG. 2B: Normal and transformed BJ cells were treated for 48 h with the indicated concentrations of a131. Selective increase in combined activity of caspase-3/7 in transformed BJ cells by a131 treatment is shown. Mean values with ±S.D. are shown (n=3). Two-tailed unpaired t tests were performed to determine statistical significance. FIG. 2C: GSEA enrichment plot and heat map of KEGG ‘cell cycle’ pathway genes in BJ cells treated with a131 for 24 h, compared to controls. The enrichment graph plots the enrichment scores for each gene (represented as bars), which are rank-ordered by their signal-to-noise metric between the DMSO control and a131 treated samples. Genes contributing to core enrichment of the pathway are highlighted with a star. The per-sample expression profiles of these genes are depicted in the heat map using an intensity-based, row-normalized color scale from blue to red, with blue indicating lower expression. Expression was generally lower in the a131-treated (dark boxes) than in the DMSO-treated cells (lighter boxes). FIG. 2D: Normal BJ cells were synchronized at the G1 phase by serum starvation (0.1% FBS) for 2 days. Subsequently, the cells were synchronously released in fresh media with 10% FBS and then treated with 5 μM a131 for 2, 4 or 11 days. After 2 or 4 days, a131 was removed and cell proliferation continued in fresh media for up to 11 days. The total number of cells at various time points were calculated using automated cell counter (SCEPTOR, Merck) and mean values with ±S.D. are shown (n>6). FIG. 2E: Normal BJ cells were treated with a131 at 2.5 and 5 μM, etoposide at 100 μM or DMSO vehicle control for 48 h and subjected to immunofluorescence analysis using anti-γ-Histone H2AX (γ-H2AX) antibody and DAPI. Note that while etoposide treatment markedly induced DNA-damage visualized by staining for γ-H2AX, a131 treatment did not do so as similar to DMSO treatment. Scale bar: 5 μm.

FIGS. 3A-3G show the selective killing effects of compound a131 by inducing centrosome de-clustering in transformed BJ cells and various cancer cell lines. FIG. 3A: Human normal and cancer cell lines were treated with a131 at a range of different concentrations (from 0.1 μM to 40 μM) for 72 h in triplicate and cell viability was determined by MTT assay. Mean concentration values for a131 to achieve 50% growth inhibition (GI₅₀) in each groups of normal and different cancer cell lines by tissue type are plotted. Mean values with ±S.D. are shown. Two-tailed unpaired t test was performed to determine the statistical significance. FIG. 3B: Indicated cancer cell lines were treated with a131 at 2.5 or 5 μM for 48 h. Cells were collected and stained with PI and subjected to FACS analysis for subG1 (<2N) population as indication of cell death. Mean values with ±S.D. are shown (n>3). FIGS. 3C-3D: Normal and transformed BJ cells stably expressing GFP-histone H2B were treated with a131 at 2.5 μM or DMSO vehicle control for 8 h and subjected to time-lapse live-cell imaging for 24 h with 5 min intervals. The duration of mitotic progression after nuclear envelope breakdown until completion of cell division in each randomly selected cell is presented in FIG. 3C (n>50 per condition). Mean values with ±S.D. are also shown from triplicated experiments. Two-tailed unpaired t test was performed to determine the statistical significance. Subsequently, cells were fixed in PFA and subjected to immunofluorescence analysis using antibodies against β-tubulin and γ-tubulin. Quantification of cells (n>50 per condition) with misaligned chromosomes is shown in FIG. 3D. Mean values with ±S.D. are shown from triplicated experiments. FIG. 3E: Indicated cancer cell lines were treated with a131 at 2.5 μM or DMSO vehicle control for 12 h and subjected to immunofluorescence analysis as in FIG. 3D and cells were counterstained with DAPI. Images were obtained using 3D-SIM super resolution microscopy. Scale bar: 5 μm. FIGS. 3F-3G: Normal and transformed BJ cells stably expressing GFP-histone H2B were treated with a131 at 2.5 μM for 24 h. Subsequently, cells were fixed in PFA and subjected to immunofluorescence analysis using antibody against β-tubulin. Images were obtained using 3D-SIM super resolution microscopy. a131 treatment caused massively misaligned chromosomes with multipolar spindles in transformed BJ cells (FIG. 3F bottom panels), but not in normal BJ cells (FIG. 3F top panels). Scale bar: 5 μm. Quantification of metaphase spindle length (pole-pole distance) (n>20 cells) in normal BJ cells is shown in FIG. 3G. Mean values with ±S.D. are shown from triplicated experiments. Two-tailed unpaired t test was performed to determine the statistical significance.

FIGS. 4A-4C show compound a131 suppresses growth of Ras-driven glioma initiating cells (GIC). FIG. 4A: Effect of a131 on murine GICs sphere growth. Representative images (upper panel) of DsRed-expressing GICs incubated for 7 days in neural stem cell medium containing DMSO, temozolomide (100 μM) or a131 (5 μM) and quantitated in the lower panel. FIG. 4B: Effect of a131 on tumor viability in murine brain explants. Immunostaining of cleaved caspase-3 (arrows positive stained) in coronal brain slices treated with DMSO or a131 (20 μM) for 4 days. Scale bar: 20 μm. FIG. 4C: Effect of a131 on tumor growth in murine brain explants. Schematic description of the experiment (top). SVZ: sub ventricular zone. Coronal slices established from the brains of tumor-bearing C57BL/6 mice before (Day 0) and after (Day 4) treatment with DMSO or a131 (20 μM). Red: DsRed-expressing GICs with scale bar: 300 μm (middle). Tumor growth ratio (Day 4/Day 0) was quantified and mean values with ±S.D. are shown from triplicate experiments (bottom). Two-tailed unpaired t-test was performed to determine the statistical significance.

FIGS. 5A-5F show inhibitory properties of various compounds against normal and transformed cells and designation into one of 4 groups. FIGS. 5A-5C: Normal and transformed BJ cells were treated with a131 and its derivatives at 5 μM for 48 h. FIG. 5A: Immunoblot analysis to determine the ability of 131 and its derivatives to induce mitotic arrest in transformed BJ cells using phospho-histone H3(Ser10) [pH3(Ser10)] and β-actin (loading control) antibodies. The fold induction of band intensities [pH3(Ser10)/β-actin] as compared to DMSO is plotted with mean values and ±S.D. (n=3). FIG. 5B: BrdU incorporation assay using normal BJ cells as in FIG. 1C. The percentage of cells with BrdU positive population in comparison with DMSO control is shown with mean values and ±S.D. (n=3). FIG. 5C: FACS analysis to determine the percentage of cells in subG1 (<2N). Mean values and ±S.D. (n=3) are shown. Indicated cells were treated with a131 and its derivatives at 5 μM (left) or at the indicated concentration (right). Note that only Group 1 compounds retain the ability to selectively kill transformed BJ cells, while compounds in Group 3 killed both normal and transformed cell lines with much less selectivity than those in Group 1. FIGS. 5D-5F: Normal and transformed BJ cells were treated with a166 at 5 μM. 48 h after treatment, cells were further treated with a159 (FIG. 5D), paclitaxel (PTX) (FIG. 5E) or etoposide (FIG. 5F) for additional 48-72 h. FACS analysis of cells stained with Annexin V together with PI. The percentage of double positive cells for Annexin V and PI is shown with mean values and ±S.D. (n=3)(FIGS. 5D-5E). FIG. 5F: MTT assay for cell viability. The results are plotted in comparison with DMSO control with mean values and ±S.D. (n=4). Where indicated, two-tailed unpaired t test was performed to determine the statistical significance.

FIGS. 6A-6B show CETSA melt curves for prominent hits with compounds a131 and a166 in normal BJ cell lysates. FIG. 6A: Normal BJ cell lysates were treated with vehicle control (DMSO) or a131 compound and then subjected to CETSA treatment. CETSA melt curves for the 16 protein hits that passed the selection criteria are shown. Curves marked with arrow represent the DMSO control treated samples and curves marked with arrow-head show the a131 treated cell lysates. FIG. 6B: Normal BJ cell lysates were treated with vehicle control (DMSO) or a166 compound and then subjected to CETSA treatment. CETSA melt curves for the 11 protein hits that passed the selection criteria are shown. Curves marked with arrow represent the DMSO control treated samples and curves marked with arrow-head show the a166 treated cell lysates. Data is presented as two individual replicates for each condition from one representative experiment. Compounds are listed in Table 4.

FIGS. 7A-7F show identification of PI5P4Ks as targets of a131 responsible for selective growth arrest in normal cells. FIGS. 7A-7B: Target identification using CETSA. All experiments were performed in two fully independent replicates. Venn diagrams of positive hits from a131 and a166 with the list of commonly targeted hits (FIG. 7A). Individual hits were ranked by distances (see materials and methods). FIG. 7B: Melting curves for PI5P4K isoforms in duplicated experiments of a131 (arrow-heads) vs. DMSO (arrows) (top) or a166 (arrow-heads) vs. DMSO (arrows) (bottom) treatment. FIGS. 7C-7D: PI5P4Ks enzyme activity assays using PI5P as substrate (see materials and methods). Inhibition curves ±S.D. (n=4) of the indicated compounds are shown. In vitro PI5P4Kα enzyme activity pre-incubated with the indicated compounds (FIG. 7C). FIG. 7D: In vivo PI5P4Ks activity using HeLa cells in order to verify cell type-independent inhibition by a131 or its indicated derivatives. FIG. 7E: BrdU incorporation assay as in FIG. 10. Normal and transformed BJ cells were transfected with control or three different sets of PI5P4Ks siRNAs (mixture of PI5P4Kα, -β, -γ) for 48 h. The percentage of cells with BrdU-positive populations is shown with mean values±S.D. (n=3). Two-tailed unpaired t tests were performed to determine statistical significance. FIG. 7F: GSEA enrichment plot and heat map of KEGG cell cycle pathway genes. Normal BJ cells were treated with a131 and a166 at 5 μM for 24 h or transfected with indicated siRNAs for 48 h. The per-sample expression profiles of these genes are depicted in the heat map using an intensity-based, row-normalized color scale from blue (−1) to red (+1), with blue indicating lower expression. DMSO and control siRNA were above zero; test compounds and siRNA were below zero.

FIGS. 8A-8F show gene expression similarity between a131 and a166 treatment and PI5P4Ks knockdown with phenocopy of the chemoprotective effects of a166. FIGS. 8A-8B: Gene expression data from normal BJ cells treated with a131 and a166 for 24 h or transfected with two different sets of PI5P4Ks for 48 h were used to identify enriched KEGG pathways. Unique and overlapping pathways were identified through separate 4-way Venn diagrams (top) and heat maps (bottom) of up- and down-regulated pathway lists, respectively. Pathways that were significant at FDR<1% were selected from each of the 4 studies and compared via the Venny software package (bioinforgp.cnb.csic.es/tools/venny). FIG. 8C: There are a total of 4 down-regulated and 12 up-regulated KEGG pathways at FDR<0.01 across all 4 experiments. In addition to cell cycle pathway as in FIG. 7F, genes contributing to core enrichment of the selected 2 pathways (DNA replication, Toll-like receptor signaling) are shown as examples of the effect for down-regulated and up-regulated pathways. The per-sample expression profiles of these genes are depicted in the heat map using an intensity-based, row-normalized color scale from blue (−2) to red (+2), with blue indicating lower expression. For DNA replication, DMSO and control siRNA were above zero; test compounds and siRNA were below zero. For TLR signaling, DMSO and control siRNA were below zero; test compounds and siRNA were above zero. FIG. 8D: Quantitative real-time PCR (qRT-PCR) analysis to measure mRNA abundance of individual PI5P4K family members in triplicated experiments. Normal and transformed BJ cells were transfected with three different sets of siRNAs to target PI5P4K isoforms as described in the materials and methods. FIGS. 8E-8F: Normal and transformed BJ cells were transfected with either control non-silencing or PI5P4Ks siRNAs for 48 h and subsequently treated with paclitaxel (PTX) (FIG. 8E) or etoposide (FIG. 8F) for additional 48-72 h. FIG. 8E: FACS analysis with PI staining. The percentage of cells in sub-G1 (<2N) is shown with mean values and ±S.D. (n=3). FIG. 8F: Caspsase-3/7 activity assay for apoptosis. The fold induction in comparison with DMSO control is plotted with mean values and ±S.D. (n=4). Where indicated, two-tailed unpaired t test was performed to determine the statistical significance.

FIGS. 9A-9D show compound a131 and Ras antagonistically control the PI3K/Akt/mTOR pathway. FIGS. 9A-9C: Immunoblot analysis of normal and transformed BJ cells treated with a131 or a166 for 24 h (FIGS. 9A, 9C) or transfected with indicated siRNAs for 48 h (FIGS. 9B, 9C). Relative ratios of phosphorylated/total levels of Akt and p70S6K are shown in comparison with DMSO. 4HT(+) indicates normal BJ cells treated with 4-OHT for 24 h to activate H-RasV12-ER. FIG. 9D: BrdU incorporation assay as in FIG. 10. 4-OHT [4HT(+)] was added in normal BJ cells for 24 h. Subsequently, cells were treated with a131 or a166 at 5 μM for 48 h (top). Normal BJ cells were transfected with indicated siRNAs for 48 h and treated with 4-OHT for 24 h (bottom). The percentage of cells with BrdU positive population in comparison with DMSO is shown with mean values and ±S.D. (n=3).

FIGS. 10A-10K show identification of positive cross-talk between Ras/Raf/MEK/ERK and PI3K/Akt/mTOR pathways via Ras-IPIK3IP1-IPI3K signaling network. FIG. 10A: The PI3K network analysis using gene expression of PI3K regulators in normal (top) and transformed BJ cells (bottom) treated with a131 at 5 μM for 24 h. PI3K regulators, including PIK3IP1, were identified to interact with PI3K with experimental support and high confidence from STRING. The per-sample expression profiles of PIK3IP1 are depicted in the heat map (left). Color: negative log FDR (false discovery rate), coded from white to dark grey in a scale from 0.15-5.37. Size: log ratio; Up-regulation (star) or down-regulation (no star); Shape: upstream (square), parallel (diamond) or downstream (circle). FIGS. 10B, 10E, 10G: qRT-PCR analysis of PIK3IP1 expression in BJ cell lines treated with a131 or a166 at 5 μM for 24 h. Mean values±S.D. (n=3). FIG. 10C: Immunoblot analysis of normal BJ cells treated with 4-OHT [4HT(+)] for 24 h to activate H-RasV12-ER and subsequently with a131 (left) or transfected with indicated siRNAs for 48 h (right). FIG. 10D: ChIP analysis of normal BJ cells treated with a131 using antibodies against RNA polymerase II (Pol II) for the PIK3IP1 gene promoter (n=3). FIG. 10E: Normal BJ cells treated with a131 were subsequently treated with MEK inhibitor U0126 (10 μM) for additional 2 h. Then, 4-OHT added to activate H-RasV12-ER for various time points. FIG. 10F: BrdU incorporation (left, n=3) and immunoblot analysis (right) of normal BJ cells transfected with indicated siRNAs for 48 h and treated with a131 for additional 24 h. FIG. 10G: Various normal and Ras-mutant cancer cell lines were treated with a166 at 0, 2.5, and 5 μM for 24 h. FIG. 10H: qRT-PCR analysis of PIK3IP1 expression (top) and immunoblot analysis (bottom) of Ras-transformed and -mutant cells treated with MEK inhibitor U0126 (10 μM) and ERK inhibitor SCH722984 (1.2 μM) for 24 h. FIG. 10I: Immunoblot analysis (top) and caspase-3/7 activation (bottom, n=3) of HCT116 cells transfected with indicated siRNAs for 48 h and treated with U0126 (10 μM) and SCH722984 (1.2 μM) for additional 24 h. FIG. 10J: Immunoblot analysis of normal BJ cells treated with PlKfyve inhibitor YM-201636 (100 nM) for 2 h and subsequently with a131 for 24 h. Relative ratios of PIK3IP1/β-actin are shown compared with DMSO. FIG. 10K: Proposed model of cross-talk between Ras/Raf/MEK/ERK and PI3K/Akt/mTOR pathway via Ras-IPIK3IP1-IPI3K signaling network for cancer cell proliferation.

Where indicated, relative ratios of phosphorylated/total levels of Akt and p70S6K are shown compared with DMSO. Two-tailed unpaired t tests were performed to determine the statistical significance.

FIGS. 11A-11E show PIK3IP1 mRNA expression in various normal and Ras- or Raf-mutant cancer cell lines and across indicated cancer-normal and cancer-cancer using TOGA dataset. FIGS. 11A, 11B, 11E: qRT-PCR analysis of PIK3IP1 mRNA expression in various normal and Ras- or Raf-mutant cancer cell lines. FIG. 11A: Endogenous PIK3IP1 mRNA expression levels. FIG. 11B: Indicated cells were treated with DMSO control or a131 at 2.5 and 5 μM for 24 h. FIG. 11C-11D: Oncomine analysis of PIK3IP gene expression. FIG. 11C: PIK3IP1 mRNA expression is suppressed in human colorectal and lung adenocarcinomas where Ras mutations and activation of Ras signaling pathways are common compared with their corresponding normal tissues or squamous cell lung carcinoma where Ras mutations are uncommon. Expression microarray results of the TOGA consortium data set were analyzed, and statistical significance was calculated using the Oncomine website (oncomine.org). FIG. 11D: Negative correlations between PIK3IP1 mRNA expression and Ras mutation status in human colorectal and lung adenocarcinomas. Box plots show differences in mRNA expression across indicated cancer-normal and cancer-cancer. Data are presented as box plot distribution (line=median value). Numbers represent samples analyzed. FIG. 11E: Pharmacological inhibition of MEK/ERK attenuates Ras- and Raf-mediated suppression of PIK3IP1. Various Ras- or Raf-mutant cancer cell lines were treated with MEK inhibitor (MEKi) U0126 and/or ERK inhibitor (ERKi) SCH772984 for 24 h and PIK3IP1 mRNA expression was measured by qRT-PCR. Of note, increase in PIK3IP1 mRNA expression was much more prominent in Raf-mutant cancer cells, suggesting the high MAPK activity is responsible for the suppression of PIK3IP1.

FIGS. 12A-12D show proposed models of effect of a131 or a166 on cell cycle progression in normal and cancer cells. Inhibition of PI5P4Ks by a131 or a166 arrests normal cells at the G1/S phase of the cell cycle by suppressing the PI3K/Akt/mTOR signaling pathway via transcriptional up-regulation of PIK3IP1 (FIG. 12A, top). This cell cycle arrest is reversible after drug removal (FIG. 12A, bottom). In contrast, mutation or activation of the Ras/Raf/MEK/ERK pathway in cancer cells promotes positive cross-talk with the PI3K/Akt/mTOR pathway by negative transcriptional regulation of PIK3IP1, which allows cancer cells to bypass a131-induced growth arrest at the G1/S phase of the cell cycle, but subsequently leads cancer cells to a131-induced mitotic catastrophe and cell death (FIG. 12B). Pre-treatment with a166 protects normal cells from chemotherapeutic toxicity by arresting normal cells at the G1/S phase of the cell cycle (FIG. 12C). In contrast, mutation or activation of the Ras/Raf/MEK/ERK pathway in cancer cells bypasses the growth arrest, leading to cell death caused by chemotherapeutic drugs (Eto, etoposide; PTX, paclitaxel) (FIG. 12D). Of note, this Ras-IPIK3IP1-IPI3K signaling network identified in this study may contribute to “oncogene collaboration” of the Ras and PI3K pathways for cancer cell growth and proliferation.

FIGS. 13A-13B show the selective killing effect of a131 is by inducing apoptosis in various cancer, but not normal cell lines. Indicated cancer and normal cell lines were treated with a131 at 2.5 or 5 μM for 48 h. Cells were collected and stained with PI (FIG. 13A) or Annexin V (FIG. 13B) according to the manufacturer's instructions (eBioscience) and subjected to FACS analysis for subG1 (<2N) population (FIG. 13A) and Annexin V positive population (FIG. 13B) as indication of cell death via apoptosis. Mean values with ±S.D. are shown (n>3). Both subG1 population and Annexin V expression are significantly higher (p<0.0001) in treated cancer cells compared to the control group treated with DMSO. Two-tailed unpaired t test was performed to determine the statistical significance. n.s.: not significant

### DETAILED DESCRIPTION OF THE INVENTION

Bibliographic references mentioned in the present specification are for convenience listed in the form of a list of references and added at the end of the examples. The whole content of such bibliographic references is herein incorporated by reference.

### Definitions

For convenience, certain terms employed in the specification, examples and appended statements are collected here.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” encompasses the more restrictive terms “consisting essentially of” and “consisting of”. The variations of the word “comprising”, such as “comprise” and “comprises”, and “including”, such as “include” and “includes”, have correspondingly varied meanings.

As used herein, “mitotic catastrophe” refers to mitotic arrest concomitant with de-clustered centrosomes and multipolar mitotic-spindles leading to cell death. The mechanism by which treatment of cells with group 1 compounds of the invention leads to, or causes, the cells to undergo mitotic catastrophe, is not clear and may be via a direct or indirect activity of said compounds.

As used herein, the term “phosphatidylinositol 5-phosphate 4-kinase family” and/or the term “(PI5P4Ks)” are intended to refer to the family of PI5P4K enzymes comprising the three isoforms PI5P4Kα, PI5P4Kβ and PI5P4Kγ. PI5P4K is also known as PIP4K2. It is known that there are sequence variants of the three main isoforms PI5P4Kα, PI5P4Kβ and PI5P4Kγ due to alternative splicing and it would be understood by the skilled person that the invention is intended to encompass modulation of sequence variants of the PI5P4Ks. The mRNA sequences and coding regions of PI5P4Kα (PIP4K2A, UniGene 138363), PI5P4Kβ (PIP4K2B, UniGene 171988) and PI5P4Kγ (PIP4K2C, UniGene 6280511) are provided in Table 1 and the sequence listings.

TABLE 1
List of human PI5P4Ks and their respective SEQ ID NO.

| Description | SEQ ID NO: |
|---|---|
| PI5P4KA variant 1 nucleotide | 19 |
| PI5P4KA variant 1 peptide | 20 |
| PI5P4KA variant 2 nucleotide | 21 |
| PI5P4KA variant 2 peptide | 22 |
| PI5P4KB nucleotide | 23 |
| PI5P4KB peptide | 24 |
| PI5P4KC variant 1 nucleotide | 25 |
| PI5P4KC variant 1 peptide | 26 |
| PI5P4KC variant 2 nucleotide | 27 |
| PI5P4KC variant 2 peptide | 28 |
| PI5P4KC variant 3 nucleotide | 29 |
| PI5P4KC variant 3 peptide | 30 |
| PI5P4KC variant 4 nucleotide | 31 |
| PI5P4KC variant 4 peptide | 32 |

As used herein, the term “small interfering RNA” or “siRNA”, sometimes known as short interfering RNA or silencing RNA, is intended to refer to a class of double-stranded RNA molecules, 20-25 base pairs in length, which function within the RNA interference (RNAi) pathway. It interferes with the expression of specific genes with complementary nucleotide sequences by degrading mRNA after transcription (Agrawal N, et al., Microbiology and Molecular Biology Reviews. 67(4): 657-685 (2003)) preventing translation. Moreover, siRNA with sequences having at least 70% identity, at least 80% identity, at least 90%, at least 95% identity, preferably at least 99% identity to the PI5P4Ks sequences may be used to inhibit PI5P4Ks expression in order to cause normal cells to enter cell cycle arrest at G1/S. It would be understood that there are software systems available to assist in siRNA design to minimize off-target effects.

The term “subject” is herein defined as vertebrate, particularly mammal, more particularly human. For purposes of research, the subject may particularly be at least one animal model, e.g., a mouse, rat and the like. For treatment of hyperproliferation disorders, however, the subject may be a human with cancer cells.

The term “treatment”, as used in the context of the invention refers to prophylactic, ameliorating, therapeutic or curative treatment.

The term “transformed cells” is herein intended to refer to engineered cells, such as those described herein which were tested for screening the initial compounds. Cells used in the Examples were transformed with Ras, hTer, p53_ko and RB_ko and grow in an anchorage independent fashion.

The term “hyperproliferative cells” is herein intended to generally include naturally occurring cancer cell lines. Transformed cells are not necessarily the same as hyperproliferative cells.

As used herein, the term “variant”, refers to one or more changes to a compound structure that has little or no detrimental effect on the ability of the compound to modulate the activity of at least one phosphatidylinositol 5-phosphate 4-kinase (PI5P4K) family member and/or at least one phosphoinositide 3-kinase-interacting protein 1 (PIK3IP1). For example, it is within the purview of a skilled person to generate a structural variant of [5-((E)-2-(1H-indol-3-yl)vinyl)isoquinoline] which retains a useful degree of PI5P4K inhibitory activity.

According to a preferred aspect, the present invention provides a method for modulating cell survival, comprising contacting at least one cell with at least one phosphatidylinositol 5-phosphate 4-kinase family (PI5P4Ks) modulator and/or at least one phosphoinositide 3-kinase-interacting protein 1 (PIK3IP1) modulator.

In a preferred embodiment, the at least one modulator inhibits PI5P4Kα, PI5P4Kβ and/or PI5P4Kγ activity and/or activates PIK3IP1 to cause cell cycle arrest at G1/S in normal cells, but not in transformed or hyperproliferative cells.

In another preferred embodiment, the at least one modulator inhibits PI5P4Kα, PI5P4Kβ and PI5P4Kγ activity. Preferably, the PI5P4Kα, PI5P4Kβ and/or PI5P4Kγ are human.

Activation of PIK3IP1 expression may be achieved by pharmacological inhibition of MEK and/or ERK. It has been demonstrated herein that inhibition of MEK and ERK significantly increased PIK3IP1 expression and caused reversible growth arrest in normal cells.

In another preferred embodiment, if said at least one modulator inhibits PI5P4K activity and causes normal cells to enter cell cycle arrest at G1/S, the at least one modulator is chemoprotective for said normal cells. In the present application compounds referred to as being in Group 1 and/or Group 2 are examples of such modulators. Structural formulae of non-limiting examples are shown in Table 3.

In the present application, compounds of Group 1 cause mitotic catastrophe in transformed or hyperproliferating cells and have chemotherapeutic activity.

In another preferred embodiment, if said at least one modulator inhibits PI5P4K activity and causes cell cycle arrest at G1/S in normal cells but not in transformed or hyperproliferative cells, the modulator is chemoprotective for said normal cells. In the present application compounds of Group 2 are examples of such modulators.

In another preferred embodiment said transformed or hyperproliferative cells are cancer cells.

In another aspect of the invention, there is provided a composition comprising at least one compound defined according to any aspect of the invention for use in chemoprotection of normal cells and/or chemotherapy of transformed or hyperproliferating cells.

According to a preferred embodiment, the at least one compound is a Group 1 or Group 2 compound or variant thereof or at least one siRNA that inhibits PI5P4Kα, PI5P4Kβ and/or PI5P4Kγ.

According to any aspect of the present invention, the amino acid sequences of PI5P4Kα variants are represented by SEQ ID NO: 20 and 22; the amino acid sequence of PI5P4Kβ is represented by SEQ ID NO: 24; and the amino acid sequences of PI5P4Kγ variants are represented by SEQ ID Nos 26, 28, 30 and 32.

According to any aspect of the present invention, the nucleic acid sequences of PI5P4Kα variants are represented by SEQ ID NO: 19 and 21; the nucleic acid sequence of PI5P4Kβ is represented by SEQ ID NO: 23; and the nucleic acid sequences of PI5P4Kγ variants are represented by SEQ ID Nos 25, 27, 29 and 31. It would be understood that inhibitory siRNA may be directed to any suitable region of the PI5P4Kα, PI5P4Kβ and/or PI5P4Kγ nucleic acid sequences.

Efficient siRNA-mediated gene silencing requires selection of a sequence that is complementary to the intended target and possesses sequence and structural features that encourage favourable functional interactions with the RNA interference (RNAi) pathway proteins. Considerations in selection of an optimized sequence are known to the skilled person (for example, Angart P. A. et al., Nucleic Acid Therapeutics. 26(5), 309-317 (2016); Agrawal N, et al., Microbiology and Molecular Biology Reviews. 67(4): 657-685 (2003)).

In another preferred embodiment, the at least one siRNA is selected from the group comprising SEQ ID Nos 1-8.

According to another aspect of the invention there is provided a method for identifying compounds that modulate PI5P4Ks activity and are suitable for use in treating a hyperproliferative disorder or disease, said method comprising:

(a) providing at least one cell comprising said PI5P4Ks;

(b) contacting said at least one cell with at least one test compound;

(c) detecting whether PI5P4Kα, PI5P4Kβ and/or PI5P4Kγ activity is inhibited and the at least one cell enters cell cycle arrest at G1/S phase, and comparing with untreated cells.

If the at least one test compound inhibits PI5P4Kα, PI5P4Kβ and/or PI5P4Kγ activity and causes cell cycle arrest at G1/S phase in normal cells, said at least one test compound is chemoprotective for normal cells during chemotherapy.

According to a preferred embodiment, inhibition of PI5P4Kα, PI5P4Kβ and/or PI5P4Kγ activity up-regulates PIK3IP1 and inhibits the PI3K/Akt/mTOR pathway in said normal cells.

According to the invention it has been found that Ras activation in transformed or hyperproliferative cells suppresses PIK3IP1 expression and its up-regulation by PI5P4Ks inhibition, thereby counteracting PI5P4Ks inhibition-induced suppression of the PI3K/Akt/mTOR pathway in said transformed or hyperproliferative cells.

According to a preferred embodiment of the method of the invention, said transformed or hyperproliferative cells are Ras-activated cancer cells.

According to another preferred embodiment of the method of the invention, the at least one test compound is a small molecule, aptamer or siRNA. Preferably, said siRNA is directed to a portion of the DNA sequence of at least one PI5P4K isoform. More preferably, said at least one PI5P4K isoform is selected from the group PI5P4Kα, PI5P4Kβ and PI5P4Kγ. Examples of such target DNA sequences are shown in SEQ ID Nos: 19, 21, 23, 25, 27, 29 and 31. More specific target sequences are shown in Example 1 as SEQ ID Nos: 1-8.

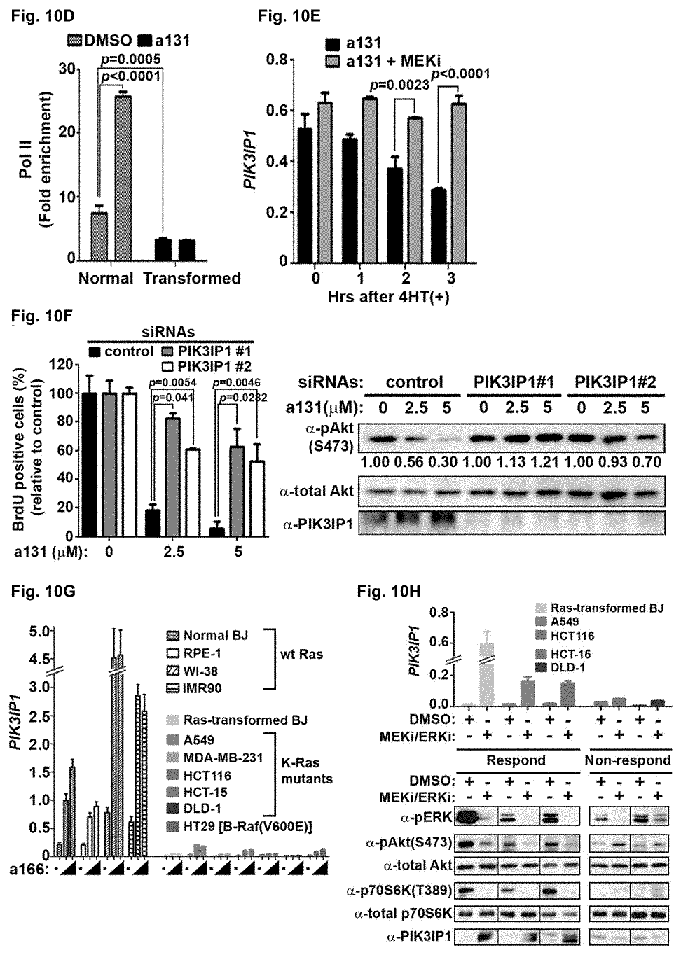

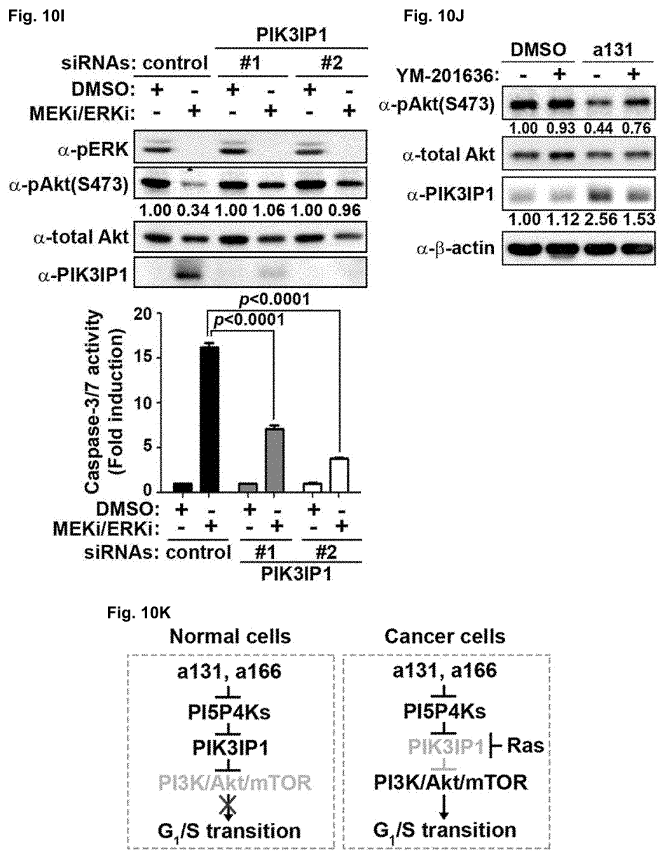

According to another preferred embodiment of the method of the invention, inhibition of PI5P4K activity is indicated by an up-regulation of PIK3IP1 at both the mRNA and protein levels compared to untreated cells. PIK3IP1 may also be up-regulated by the administration of MEK and/or ERK inhibitors, for example U0126 and SCH722984, respectively, as described in Example 8 and shown in FIGS. 10H-10I.

According to another aspect of the invention there is provided a method of treatment of a hyperproliferative disease or disorder, which method comprises the administration of an effective amount of at least one compound which inhibits PI5P4Kα, PI5P4Kβ and/or PI5P4Kγ activity and causes cell cycle arrest at G1/S in normal cells but not in transformed or hyperproliferating cells, and an effective amount of an anti-hyperproliferation agent.

According to a preferred embodiment, said transformed or hyperproliferating cells are Ras-activated cancer cells.

According to another preferred embodiment, said anti-hyperproliferation agent is a chemotherapeutic agent.

According to any aspect of the present invention, said at least one compound is a small molecule, aptamer or siRNA.

According to another preferred embodiment, said at least one compound is, for example, [5-((E)-2-(1H-indol-3-yl)vinyl)isoquinoline], or a variant thereof.

According to another preferred embodiment, said at least one compound is, for example, at least one siRNA directed to a DNA target sequence selected from the group comprising SEQ ID NO: 1 to SEQ ID NO: 8. More preferably, the at least one siRNA target sequence is selected from the group comprising SEQ ID NO: 1 to 3; SEQ ID NO: 3 to 5 and SEQ ID NO: 6 to 8.

According to another preferred embodiment, said at least one compound has at least a second activity whereby it further causes transformed or hyperproliferating cells to undergo mitotic catastrophe and is, for example, (Z)-2-(1H-indol-3-yl)-3-(5-isoquinolyl)prop-2-enenitrile or (Z)-3-(isoquinolin-5-yl)-2-(1-(2-(4-methylpiperazin-1-yl)acetyl)-1H-indol-3-yl)acrylonitrile, or a variant thereof. In the present application compounds of group 1 are examples of such modulators and can be considered as chemoprotective for said normal cells, although they also selectively kill transformed or hyperproliferating cells.

According to another preferred embodiment of any aspect of the invention, said cell cycle arrest at G1/S phase is transient and/or reversible.

According to another aspect of the invention there is provided a use of at least one modulator that inhibits PI5P4Kα, PI5P4Kβ and/or PI5P4Kγ activity and causes cycle arrest at G1/S phase in normal cells and wherein, additionally, said modulator causes transformed or hyperproliferating cells to undergo mitotic catastrophe for the preparation of a medicament for the treatment of a hyperproliferative disease or disorder.

According to another aspect of the invention there is provided a use of at least one modulator that inhibits PI5P4Kα, PI5P4Kβ and/or PI5P4Kγ activity and causes cell cycle arrest at G1/S phase in normal cells, for the preparation of a medicament for the treatment of a hyperproliferative disease or disorder in combination with an antiproliferative agent. Preferably said at least one modulator is selected from Group 1 and/or Group 2 compounds as described herein. Said at least one modulator may alternatively or additionally be any suitable siRNA or aptamer.

In a preferred embodiment the at least one siRNA that inhibits PI5P4Kα, PI5P4Kβ and/or PI5P4Kγ activity is directed to a DNA target sequence selected from the group comprising SEQ ID Nos: 19, 21, 23, 25, 27, 29 and 31, more particular examples of which are represented by the group SEQ ID NO: 1 to SEQ ID NO: 8.

According to a preferred embodiment, use of the at least one modulator leads to an increase in the expression of PIK3IP1 in normal cells.

According to another preferred embodiment, the hyperproliferative disease or disorder involves Ras-activated cancer cells.

Compounds of the present invention will generally be administered as a pharmaceutical formulation in admixture with a pharmaceutically acceptable adjuvant, diluent or carrier, which may be selected with due regard to the intended route of administration and standard pharmaceutical practice. Such pharmaceutically acceptable carriers may be chemically inert to the active compounds and may have no detrimental side effects or toxicity under the conditions of use. Suitable pharmaceutical formulations may be found in, for example, Remington The Science and Practice of Pharmacy, 19th ed., Mack Printing Company, Easton, Pa. (1995). For parenteral administration, a parenterally acceptable aqueous solution may be employed, which is pyrogen free and has requisite pH, isotonicity, and stability. Suitable solutions will be well known to the skilled person, with numerous methods being described in the literature. A brief review of methods of drug delivery may also be found in e.g. Langer R., Science 249: 1527-33 (1990).

Otherwise, the preparation of suitable formulations may be achieved routinely by the skilled person using routine techniques and/or in accordance with standard and/or accepted pharmaceutical practice.

The amount of a compound in any pharmaceutical formulation used in accordance with the present invention will depend on various factors, such as the severity of the condition to be treated, the particular patient to be treated, as well as the compound(s) which is/are employed. In any event, the amount of a compound in the formulation may be determined routinely by the skilled person.

For example, a solid oral composition such as a tablet or capsule may contain from 1 to 99% (w/w) active ingredient; from 0 to 99% (w/w) diluent or filler; from 0 to 20% (w/w) of a disintegrant; from 0 to 5% (w/w) of a lubricant; from 0 to 5% (w/w) of a flow aid; from 0 to 50% (w/w) of a granulating agent or binder; from 0 to 5% (w/w) of an antioxidant; and from 0 to 5% (w/w) of a pigment. A controlled release tablet may in addition contain from 0 to 90% (w/w) of a release-controlling polymer.

A parenteral formulation (such as a solution or suspension for injection or a solution for infusion) may contain from 1 to 50% (w/w) active ingredient; and from 50% (w/w) to 99% (w/w) of a liquid or semisolid carrier or vehicle (e.g. a solvent such as water); and 0-20% (w/w) of one or more other excipients such as buffering agents, antioxidants, suspension stabilisers, tonicity adjusting agents and preservatives.

Depending on the disorder, and the patient, to be treated, as well as the route of administration, compounds may be administered at varying therapeutically effective doses to a patient in need thereof.

However, the dose administered to a mammal, particularly a human, in the context of the present invention should be sufficient to effect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the patient to be treated, and the stage/severity of the disease.

Having now generally described the invention, the same will be more readily understood through reference to the following examples which are provided by way of illustration, and are not intended to be limiting of the present invention.

### EXAMPLES

### Example 1: Methods

Standard molecular biology techniques known in the art and not specifically described were generally followed as described in Sambrook and Russel, Molecular Cloning: A Laboratory Manual, Cold Springs Harbor Laboratory, New York (2001).

### Cell Lines, Culture and Reagents

Isogenic BJ human foreskin fibroblast cell lines, including non-transformed (normal) and transformed BJ cells and all gastric cancer cell lines, were kind gifts from Dr. Mathijs Voorhoeve and Dr. Patrick Tan (Duke-NUS), respectively and tested for mycoplasma infection. The culture media for the cell lines used in this study are summarized in Table 2. All other human cancer cell lines used in this study were purchased from ATCC and cultured in accordance with ATCC's instructions. H-RasV12-ER was activated by exposing the BJ-derived fibroblasts to 4-OHT (100 nM, Sigma-Aldrich). Three different sets of siRNAs were used in this study to target PI5P4K isoform DNA sequences as follows:

pool#1,
PI5P4Kα
(5′-CTGCCCGATGGTCTTCCGTAA-3′; SEQ ID NO: 1),
PI5P4Kβ
(5′-CACGATCAATGAGCTGAGCAA-5′; SEQ ID NO: 2),
and
PI5P4Kγ
(5′-CCGGAGCAGTATGCTAAGCGA-3′; SEQ ID NO: 3);
pool#2,
PI5P4Kα
(5′-CGGCTTAATGTTGATGGAGTT-3′; SEQ ID NO: 4),
PI5P4Kβ
(5′-CCCTCGATCTATTTCCTTCTT-3′; SEQ ID NO: 5),
and
PI5P4Kγ
(5′-CCGGAGCAGTATGCTAAGCGA-3′; SEQ ID NO: 3);
pool#3,
PI5P4Kα
(5′-CCTCGGACAGACATGAACATT-3′; SEQ ID NO: 6),
PI5P4Kβ
(5′-CAAACGCTTCAACGAGTTTAT-3′; SEQ ID NO: 7),
and
PI5P4Kγ
(5′-CCGAGTCAGTGTGGACAACGA-3′; SEQ ID NO: 8).

To knockdown PIK3IP1, siRNAs #1 (5′-AGAGGCTAACCTGGAAACTAA-3′; SEQ ID NO: 9) and #2 (5′-TACACTGTTATTCATGGTTAA-3′; SEQ ID NO: 10) were used. Non-silencing control siRNA was purchased from Dharmacon. For siRNA transfection, Lipofectamine 2000 (Invitrogen) or Dharmafect (Dharmacon) was used according to the manufacturer's instructions.

### Cytotoxicity Test

Cells were plated in 96-well microplates on day 0, and a131 was added to each well on day 1 at a range of different concentrations (from 0.1 μM to 40 μM) in triplicate. After 3 days of culture, the number of viable cells was determined using MTT cell proliferation assays by adding thiazolyl blue tetrazolium bromide (MTT reagent, Invitrogen) at a concentration of 0.5 mg/mL to each well and incubating for 4 h at 37° C. The medium was then removed, and the blue dye remaining in each well was dissolved in DMSO by mixing with a microplate mixer. The absorbance of each well was measured at 540 nm and 660 nm using a microplate reader (Benchmark plus, Bio-Rad). Optical density (OD) values were calculated by subtracting the absorbance at 660 nm from the absorbance at 540 nm. Mean OD values from control cells containing only DMSO treated wells were set as 100% viable. The concentration of drug that reduced cell viability by 50% (GI₅₀) was calculated by non-linear regression fit using GraphPad Prism.

### Crystal Violet Staining

Cells were washed twice in 1×PBS and stained with 0.5% crystal violet dye (in methanol:deionized water=1:5) for 10 min. Excess crystal violet dye was removed by washing five times (10 min per wash) with deionized water on a shaker, and the culture plates were dried overnight.

### Analysis of Cell Death and Cell Growth Arrest

Cell death was assessed via Annexin V and/or PI (propidium iodide) staining according to the manufacturer's instructions (eBioscience). Cell growth arrest was assessed by direct measurement of DNA synthesis through incorporation of the nucleoside analog bromodeoxyuridine (BrdU). Briefly, BrdU (30 μM, Sigma-Aldrich) was added for 2 h before harvesting cells. Cells were subsequently stained with Pacific Blue-conjugated BrdU antibody (Invitrogen) for 1 h followed by PI staining. Stained cells were analyzed by MACSQuant (MACS). Three independent experiments were performed in triplicate. The percentage of Annexin V/PI- or BrdU-positive cells was quantified using Flow Jo software (Becton Dickinson). Where indicated, the combined activity of caspase-3/7 was determined using the caspase-Glo 3/7 Assay Kit (Promega) and normalized to the number of viable cells as determined by MTT assay.

### In Vivo Study

BALB/c athymic female nude mice (nu/nu, 5-7 weeks) (InVivos) were kept under specific pathogen-free (SPF) conditions. The care and use of mice was approved by the Duke-NUS IACUC in accordance with protocol 2015/SHS/1030. HCT15 human colon cancer cells (5×10⁶) or MDA-MB-231 human breast cancer cells (4×10⁶ with Matrigel) were subcutaneously injected into the flanks of mice. When the mean tumor volume reached 100-300 mm³ (Day 1), the mice were randomly divided into experimental groups of 6 mice by an algorithm that moves animals around to achieve the best case distribution to assure that each treatment group has similar mean tumor burden and standard deviation. No statistical method was used to predetermine sample size. The animals were treated with either intraperitoneal (IP) or oral (PO) injection of a131 (20 mg/kg), b5 (40-80 mg/kg) or vehicle control twice per day for 12 days (HCT115) or 15 days (MDA-MB-231). Compounds a131 and b5 were dissolved in DMSO followed by the addition of PEG400 and deionized water (pH 5.0) (final concentrations, 10% DMSO, 50% PEG400). Paclitaxel (Cayman Chemical) was dissolved in ethanol:Tween 80=1:3 (v/v) solution followed by the addition of a 5% glucose solution (final ratio, ethanol:Tween 80:5% glucose=5:15:80) and injected via the tail vain (IV). Tumor dimensions were measured using calipers, and tumor volume (mm³) was calculated using the formula width²×length/2 in a blinded fashion. On Day 12 (HCT15) or Day 16 (MDA-MB-231), mice were sacrificed. Tumors were collected, fixed overnight in 4% paraformaldehyde (PFA) and stored in 70% ethanol. For immunostaining, antigens were retrieved from formaldehyde-fixed, paraffin-embedded tumor tissue sections for 30 min by boiling in sodium citrate buffer (pH 6.0) using a microwave histoprocessor (Milestone). Endogenous peroxidase activity in tissue sections was depleted by treatment with 3% hydrogen peroxide (H₂O₂ in 1×TBS) for 20 min at room temperature. Tumor tissue sections were incubated overnight with anti-β-tubulin (Abcam; 1:100 in 3% BSA/TBS-Tween 20) at 4° C. followed by incubation with goat anti-rabbit FITC-conjugated secondary antibody (Invitrogen; 1:200 in 3% BSA/TBS) for 1 h at 25° C. After dehydration treatment, coverslips were mounted using DAPI mounting medium (Vector). Images were acquired in 3D-SIM mode using a super resolution microscope (Nikon), and the number of cells with either 2 or mitotic-spindles was quantified (n>50 cells per section, 6-7 sections per treatment). For detection of apoptosis using the TUNEL method, the ApopTag Plus Peroxidase In Situ Apoptosis Detection Kit (Millipore) was used for formaldehyde-fixed, paraffin-embedded tumor tissue sections treated with b5 (80 mg/kg, IP) or control vehicle for 12 days. Slide scans were acquired using a MetaSystems Metafer built on a Zeiss Axiolmager Z.2 upright microscope. The system is equipped with a CoolCubel camera and a Zeiss Plan-Neofluar 20×/0.5 Ph2 objective lens. Image acquisition was controlled with Metafer4 software, and stitching was performed with the VSlide software and further processed using the open source software FIJI. A custom macro was used to batch process the images in the following sequence: Gaussian filter, color deconvolution of hematoxylin and DAB, thresholding the hematoxylin image, watershed to separate touching nuclei, and then count the number of hematoxylin-stained nuclei. For the DAB image, a fixed threshold was used, watershed applied and the number of DAB stained nuclei counted. In all cases, no data or animals were excluded and results are expressed as mean and standard deviation of the mean.

### Sphere Formation Assay

Murine glioma-initiating cells (GICs) were established and cultured as described previously (Saga, I. et al., Neuro. Oncol. 16: 1048-1056 (2014)). Briefly, Ink4a/Arf-null neural stem/progenitor cells were transduced with human H-RasV12 and DsRed and propagated in serum-free Dulbecco modified Eagle medium (DMEM)/F12 (Sigma-Aldrich) supplemented with recombinant epidermal growth factor (EGF; PeproTech) and basic fibroblast growth factor (PeproTech) at 20 ng/ml, heparan sulfate (Sigma-Aldrich) at 200 ng/ml and B27 supplement without vitamin A (Invitrogen, Carlsbad, Calif.). GICs were dissociated and plated in 96-well plates at a density of 100 cells/well. Vehicle (DMSO), temozolomide (Sigma-Aldrich) at 100 μM or a131 at 5 μM were added and sphere formation and size evaluated 7 days after plating. Three plates were prepared for each treatment group, and 30 wells were quantified per plate. Images were acquired on a BZ-X700 inverted fluorescence microscope (Keyence). Quantification was performed by Nikon NIS-element software (n=90).

### Brain Slice Explants and Drug Treatment

Fifty thousand GICs were orthotopically implanted into the forebrain of wild-type mice, and at 7 days post-implantation, brain slice explants were established as previously described (Sampetrean, O. et al., Neoplasia 13: 784-791 (2011)). Coronal slices (200 μm) were cultured on Millicell-CM culture plate inserts (Millipore) and treated with vehicle or a131 for 4 days. Images were acquired on an FV10i Olympus confocal microscope (Olympus) and tumor area was quantified by Nikon NIS-element software. Experiments were performed in triplicate. At the end of the experiment (Day 4), slices were fixed overnight in 4% paraformaldehyde, embedded in paraffin and then sectioned at a thickness of 4 μm. Deparaffinized sections were stained with rabbit polyclonal antibody against cleaved caspase 3 (Cell Signaling). Immune complexes were detected using Histofine (Nichirei Biosciences) and ImmPACTDAB (Vector Laboratories). All animal experiments were performed in accordance with the animal care guidelines of Keio University.

qRT-PCR

Total RNA was isolated from cultured cells using the RNeasy mini kit (Qiagen). cDNA was synthesized from 1 μg of total RNA using the iScript cDNA synthesis kit (Bio-Rad). qRT-PCR analysis was performed using the iQ SYBR Green Super mix (Bio-Rad) using the following gene-specific primers:

human PI5P4Kα (5′-AAGAAGAAGCACTTCGTAGCG-3′; SEQ ID NO: 11, 5′-ATGGCTCAGTTCATTGATCGAG-3′; SEQ ID NO: 12),
human PI5P4Kβ (5′-CCACACGATCAATGAGCTGAG-3′; SEQ ID NO: 13, 5′-TCCTTAAACTTAAAGCGGCTGG-3′; SEQ ID NO: 14),
human PI5P4Kγ (5′-CCGGGAAGCCAGCGATAAG-3′; SEQ ID NO: 15, 5′-AGCTGCACTAGAAACTCCACA-3′; SEQ ID NO: 16) and
human PIK3IP1 (5′-GCTAGGAGGAACTACCACTTTG-3′; SEQ ID NO: 17, 5′-GATGGACAAGGAGCACTGTTA-3′; SEQ ID NO: 18). The TATA-binding protein (TBP) gene was used for normalization. All PCR reactions were performed in triplicate.

### Microarray Data Analysis

Biotin-labeled cRNA was prepared from 250-500 ng of total RNA using the Illumina TotalPrep RNA Amplification Kit (Ambion Inc.). cRNA yields were quantified with a Agilent Bioanalyzer, and 750 ng of biotin-labeled cRNA was hybridized to the Illumina HT-12 v4.0 Expression Beadchip according to manufacturer's instructions (Illumina, Inc.). Following hybridization, bead chips were washed and stained with Cy3-labeled streptavidin according to the manufacturer's protocol. Dried bead chips were scanned on the Illumina BeadArray Reader confocal scanner (Illumina, Inc.). Gene expression signals obtained after chip scanning were quantile normalized in Partek Genomics Suite v6.6 (Partek Inc.). Genes with a normalized maximum average signal <100 in all groups were considered similar to background and removed from further analysis. Sample outliers were detected via principal component analysis in Partek. Differentially expressed genes were identified via 1-way ANOVA with post-hoc contrasts specifying the desired pair-wise comparisons. The magnitude of differential gene expression between 2 groups was expressed as the logarithm of the fold-change (base 2), and the statistical significance of differences in gene expression were ascertained by the false discovery rate (FDR). For most analyses, genes with an absolute log fold-change >0.58 and FDR<5% were considered significantly differentially expressed. Gene expression profiles across comparator groups were visualized through heat-maps generated via the gplots library in R 3.2.3 using the gplots and RColorbBrewer packages, with genes in rows and treatments in columns. Enrichment graph plots for each gene (represented as bars), which are rank-ordered by their signal-to-noise metric between the control and treated compounds or PI5P4Ks knockdown samples. Gene expression values were row-normalized and mapped to a color scale representing an ascending scale of expression signals. In some analyses, the gene expression matrix was subjected to hierarchical clustering by Ward's algorithm (Ward, J. H. Journal of the American Statistical Association 58: 236-244 (1963)) prior to the generation of heat maps. To evaluate the effects of differential gene expression on biological mechanisms, we performed gene-set enrichment analysis (GSEA) (Subramanian, A. et al., Proc. Natl. Acad. Sci. U.S.A 102: 15545-15550 (2005)) using a customized version of the KEGG pathway repository obtained from the Molecular Signatures Database, MSigDB (Kanehisa, M. & Goto, S. KEGG: Nucl. Acids Res. 28: 27-30 (2000)). Biological pathways containing 10-200 genes were considered for analysis, and pathways with FDR<10% were considered statistically significant.

### Immunoblot Analysis

Total cell lysates were prepared with 1% triton lysis buffer [25 mM Tris HCl (pH 8.0), 150 mM NaCl, 1% triton-X100, 1 mM dithiothreitol (DTT), protease inhibitor mix (Complete Mini, Roche) and phosphatase inhibitor (PhosphoStop, Roche)] and subjected to SDS-PAGE. The following antibodies were used for immunoblotting: anti-β-actin (Sigma-Aldrich), anti-cleaved PARP (Abcam, #ab32064), anti-PI5P4Kα (#5527), anti-PI5P4Kβ (#9694), anti-cleaved caspase-3 (#9664), anti-pHistone H3(Ser10) (#3377), anti-pAkt(S473) (#9271), anti-pAkt(T308) (#13038), anti-total Akt (#9272), anti-p70S6K(T389) (#9234), anti-total p70S6K (#9202), anti-pErk (#4370), anti-γ-Histone H2AX (#9718) (Cell signaling) and anti-PIK3IP1 (Proteintech, #16826-1-AP). The secondary antibodies used were sheep anti-mouse IgG HRP and donkey anti-rabbit IgG HRP (Amersham; 1:2000 dilution). Immunoreactive proteins were visualized using ECL reagent (Amersham). Where indicated, intensities of protein bands were quantified by densitometry (Odyssey V3.0), normalized to their loading controls and then calculated as fold expression change relative to DMSO control.

### Immunofluorescence and Time-Lapse Live-Cell Imaging

For immunofluorescence analysis, cells grown on coverglass-bottom chamber slides (Lab-Tek) were fixed with 4% PFA (paraformaldehyde) for 15 min at 25° C. The fixed cells were permeabilized with 0.5% Triton X-100 and exposed to TBS containing 0.1% Triton X-100 and 2% BSA (AbDil). The following primary antibodies were diluted in PBS containing 1% BSA and 0.1% Triton X-100: anti-γ-tubulin (Sigma-Aldrich, #T6557; 1:1000) and anti-β-tubulin (Abcam, #ab18207; 1:2000). Isotype-specific secondary antibodies (1:500 dilution) coupled to Alexa Fluor 488, 594, or Cy5 (Molecular Probes) were used. Cells were counterstained with DAPI (Thermo Scientific). Images were acquired at RT with 3D-SIM mode using a Super Resolution Microscope (Nikon) equipped with an iXon EM+885 EMCCD camera (Andor) mounted on a Nikon Eclipse Ti-E inverted microscope with a CFI Apo TIRF (100×/1.40 oil) objective and processed with the NIS-Elements AR software. For time-lapse live-cell analysis, a Stage Top Incubator with Digital CO₂ mixer (Tokai) was used, and images were acquired at 37° C.

### The Cellular Thermal Shift Assay (CETSA)

Target identification was performed by cellular thermal shift assay (CETSA) coupled with quantitative mass spectrometry. In brief, normal BJ cells were lysed by combination of freeze/thaw and mechanical shearing with needle in buffer [50 mM HEPES (pH 7.5), 5 mM β-glycerophosphate, 0.1 mM Sodium Vanadate, 10 mM MgCl₂, 1 mM TCEP and 1× Protease inhibitor Cocktail]. The cell debris was removed by centrifugation at 20,000 g for 20 min at 4° C. Cell lysates were incubated with 100 μM a131, a166 or DMSO for 3 min at room temperature. Each lysate was divided into 10 aliquots for heat treatment at the respective temperatures for 3 min in a 96-well thermocycler, followed by 3 min at 4° C. The lysate was centrifuged at 20,000 g for 20 min at 4° C. and the supernatant was transferred into microtubes for MS sample preparation.

### MS-Sample Preparation

After lysis, at least 100 μg of the protein (measured with a BCA assay) was subjected to reduction, denaturation and alkylation. Samples were subsequently incubated with sequencing grade LysC (Wako) and trypsin (Promega) for digestion overnight at 37° C. The digested samples were dried using a centrifugal vacuum evaporator, solubilized in 100 mM TEAB. For each run, 25 μg of the digested protein was labeled for 1 h with 10plexTMT (Pierce). The samples were then quenched with 1M Tris buffer, pH 7.4. The labeled samples were then pooled together and desalted using a C18 Sep-Pak (Waters) cartridge and the samples were pre-fractionated into 80 fractions using a High pH reverse phase Zorbax 300 Extend C-18 4.6 mm×250 mm (Agilent) column and liquid chromatography AKTA Micro (GE) system.

### LC-MS Analysis

The fractions from the pre-fractionation were pooled into 20 fractions and pooled fractions from each experiment were subjected to mass spectrometry analysis using reverse phase liquid chromatography Dionex 3000 UHPLC system combined with Q Exactive mass spectrometer (Thermo Scientific). The following acquisition parameters were applied: Data Dependent Acquisition with survey scan of 70,000 resolution and AGC target of 3e6; Top12 MS/MS 35,000 resolution (at m/z 200) and AGC target of 1e5; Isolation window 1.6 m/z. Peak lists for subsequent searches were generated using Mascot 2.5.1 (Matrix Science) and Sequest HT (Thermo Scientific) in Proteome Discoverer 2.0 software (Thermo Scientific). The reference protein database used was the concatenated forward/decoy Human-HHV4 Uniprot database.

### Hit Selection and Ranking

Proteins with a high plateau at the highest temperature points were deleted using a cut-off at >0.3 for the average reading of the last 3 temperature points in the control (DSMO-treated) condition (Savitski, M. M. et al., Science 346 (6205): 55 (2014)). Proteins for which a low temperature plateau was not present were deleted using a cut off >0.85 for the average reading of the first three temperature points (in our experience proteins melting already at −37° C. are more prone to give false positives in a shift analysis). Euclidean distance (ED) score of thermal shifts of all the proteins with complete replicates were then calculated and ED hit lists were generated for a131 and a166 using a cut-off at median+2.75*MAD (median absolute deviation). ΔTm value of thermal shifts were calculated as average deviations between control and treated samples at 0.5 fold change, and proteins with significant positive ΔTm value of median+2.75*MAD were selected. The proteins with both significant ED score and significant ΔTm value were selected as the final hit lists corresponding to 16 and 11 proteins for a131 and a166, respectively. Melting curves which are flat and have a high plateau at the high temperature edge are less likely to correspond to direct binding (Mayer, I. A. & Arteaga, C. L. Annu. Rev. Med. 67:11-28 (2016)) and optical inspection suggest that e.g. Arsenate methyl transferase, albeit giving one of the largest ΔTm, is less likely to be a significant hit corresponding to direct target binding. The analysis steps including protein melting curve plotting, hits selection and ranking were automated using an in-house-developed script using R programming language (Core_Team, R. R: (2014)).

### PI5P4K Enzyme Assay

HeLa cells were treated with DMSO or compounds for 24 h. Cells were lysed with RIPA buffer (Sigma-Aldrich), and total protein concentrations were measured using a bicinchoninic acid protein assay kit (Thermo Scientific). Next, 10 μg of cell lysate was incubated with 1 μM PI(5)P and 500 nM ATP for 1 h at 37° C. PI5P4K activity was measured by recording luminescent signals (Tecan) using a PIP4KII Activity Assay Kit (Echelon) according to the manufacturer's instructions. For cell-free PI5P4Kα activity assays, serial dilutions of compounds were pre-incubated with 1 ng of PI5P4Kα (kind gift from Daiichi Sankyo Co., Ltd. and Daiichi Sankyo RD Novare Co., Ltd.) in reaction buffer [50 mM HEPES (pH 7.0), 13 mM MgCl₂, 0.005% CHAPS, 0.01% BSA, 2.5 mM DTT] for 1 h at 25° C. DOPS (80 μM, Avanti polar lipids), PI(5)P (20 μM, Echelon) and ATP (10 μM, Sigma-Aldrich) were added and further incubated for 90 min at room temperature. PI5P4Kα activity was measured by recording luminescent signals (Tecan) using an ADP-Glo Kinase Assay (Promega) according to the manufacturer's protocol.

### ChIP

Chromatin Immunoprecipitation (ChIP) assays were carried out using the Magna ChIP A/G Kit (Millipore) according to the manufacturer's instructions. Enrichment of PollI binding to PIK3IP1 was evaluated by qPCR using 1/10 of the immunoprecipitated chromatin as template and iQ SYBR Green Super mix (Bio-Rad). Primer sequences are available upon request.

### Example 2: Identification of Cancer-Selective Compounds

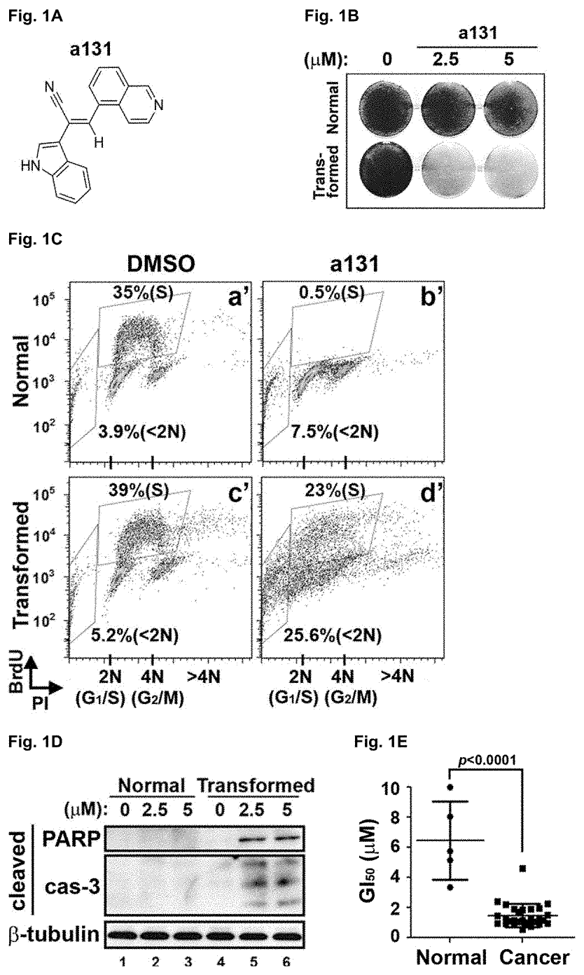

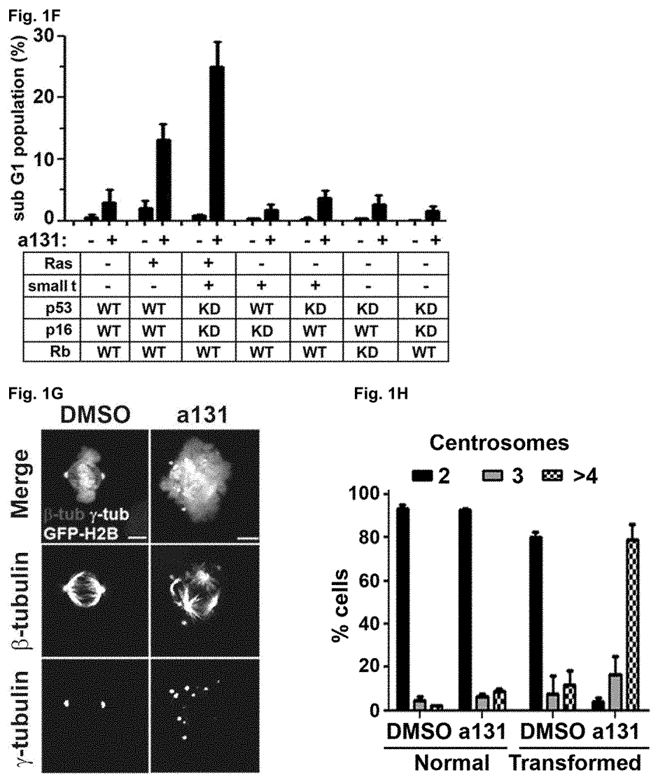

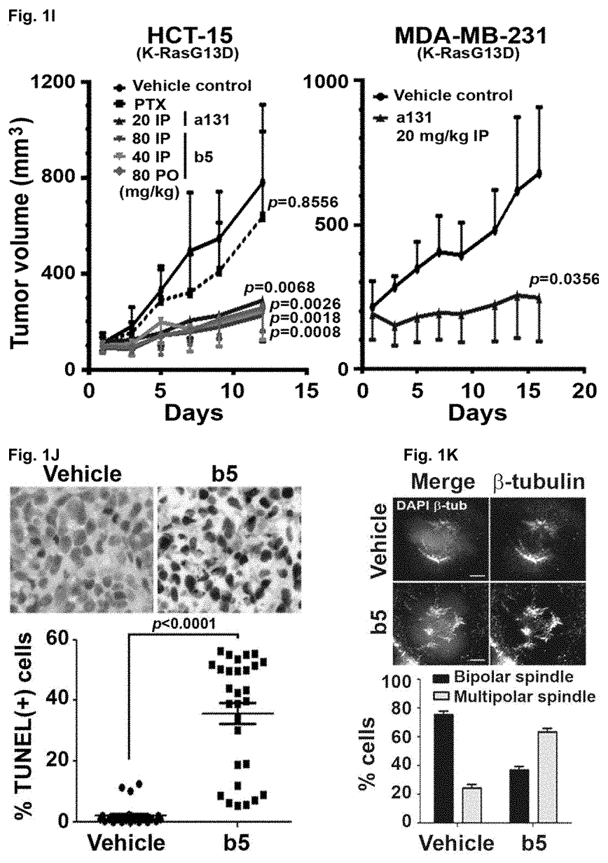

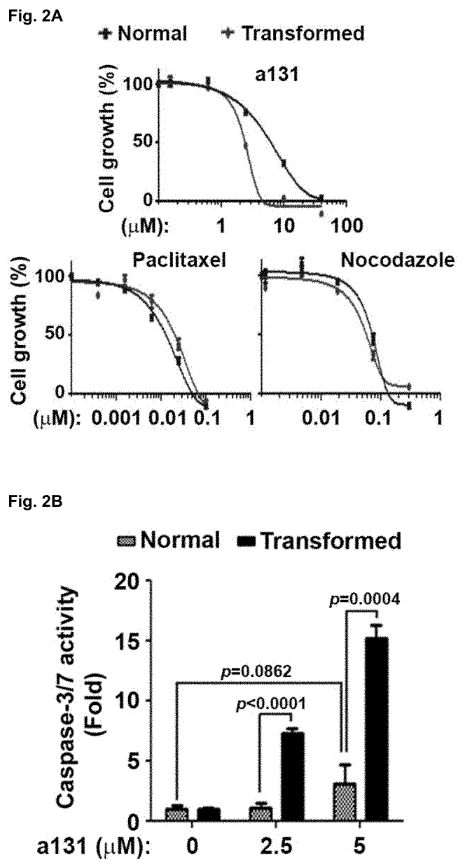

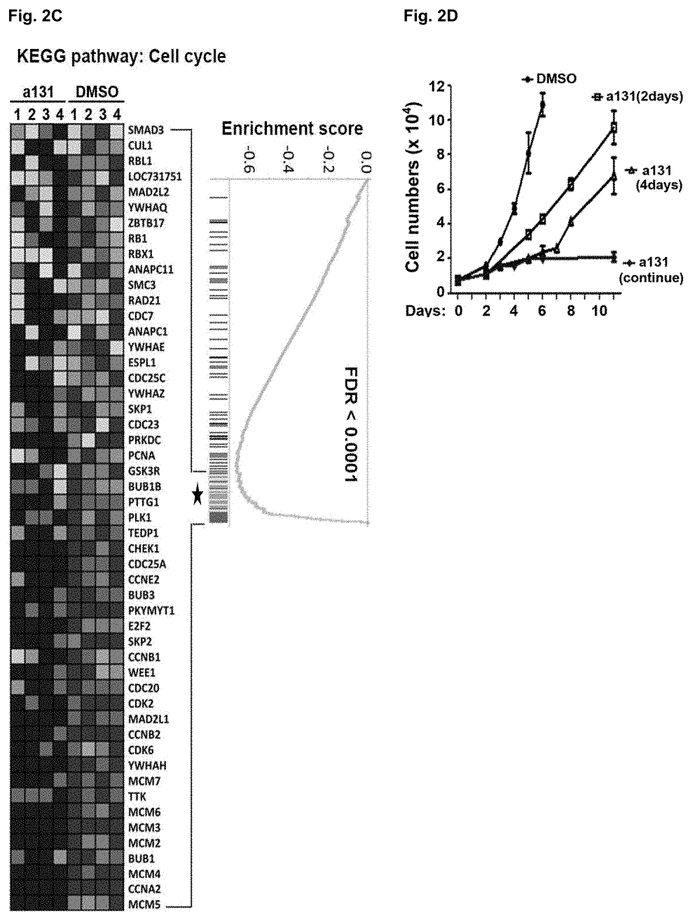

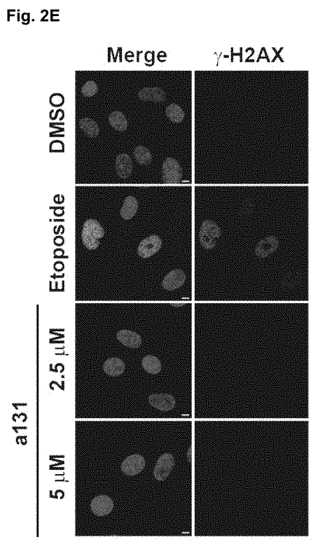

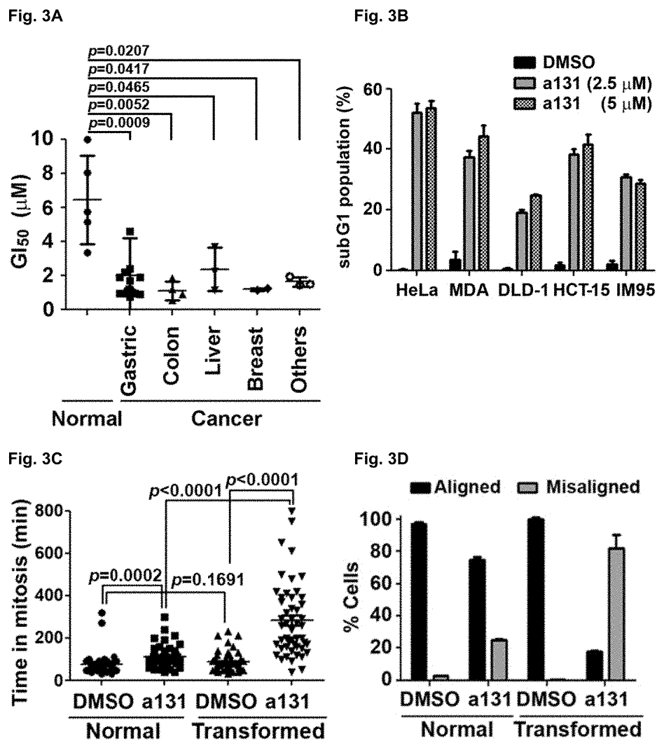

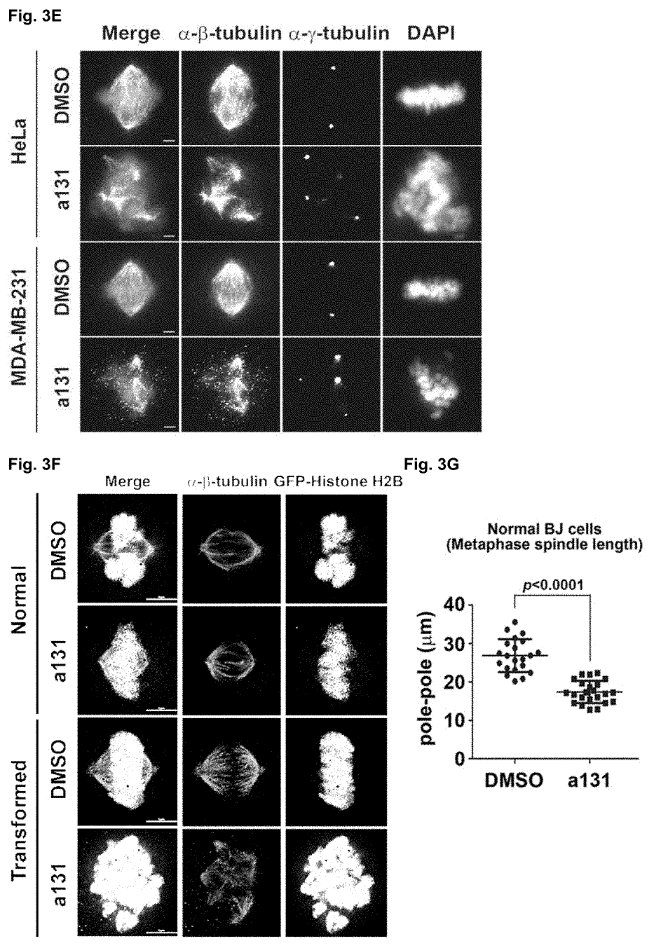

A small-molecule screen was undertaken to investigate the specific signaling networks needed for the proliferation and survival of transformed cells using isogenic human BJ foreskin fibroblasts either immortalized with only hTert (hereafter named as normal BJ) or fully transformed with hTert, small t, shRNAs against p53 and p16 and H-RasV12-ER (estrogen receptor-fused H-Ras bearing the activating G12V mutation) (hereafter named as transformed BJ). One of the screened compounds (anti-cancer compound 131; hereafter referred to as a131) (FIG. 1A) efficiently killed transformed BJ cells but not normal counterparts (FIG. 1B; FIG. 2A). In contrast, treatment with paclitaxel (microtubule stabilizer) and nocodazole (microtubule destabilizer) showed minimal selectivity (FIG. 2A). FACS analysis of the cell cycle revealed that a131 dramatically induced cell death (<2N) through apoptosis (FIG. 1D; FIG. 2B) only in transformed BJ cells and not in normal counterparts (FIG. 10). Moreover, a131 treatment significantly induced aneuploidy (>4N) only in transformed BJ cells (FIG. 10, panel d′). Instead, a131 arrested normal BJ cells at the G₁/S phase of the cell cycle with few BrdU incorporation (FIG. 10, panel b′), which was also confirmed with gene set enrichment analysis (GSEA) of genes promoting the cell cycle (FIG. 2C). Importantly, this a131-induced growth arrest in normal BJ cells was transient and reversible after a131 removal (FIG. 2D). Further, unlike the DNA-damaging Topo II inhibitor etoposide, a131-induced growth arrest occurred in the absence of genotoxic stresses (FIG. 2E). This cancer-selective lethality of a131 was further confirmed using a panel of human normal and cancer cell lines [GI₅₀=6.5 vs. 1.7 μM (normal vs. cancer)] (FIG. 1E; FIGS. 3A and 3B; Table 2), suggesting a131 is a potent antiproliferative agent with a clear selectivity toward cancer cells killing.

TABLE 2
List of normal and cancer cell lines and culture media used in this study with
the concentrations of a131 that reduced cell viability by 50% (GI50).

| normal | | | | cancer | | | |
|---|---|---|---|---|---|---|---|
| | | | culture | | | | culture |
| tissue origin | name | GI50 | media | tissue origin | name | GI50 | media |
| 1 skin | BJ normal | 8.1 | DMEM | 1 gastric | NCC24 | 1.2 | RPMI |
| 2 lung | IMR90 | 5.8 | DMEM | 2 gastric | IST1 | 2.2 | RPMI |
| 3 retina | ARPE-19 | >10 | DMEM/F12 | 3 gastric | HGC27 | 1.2 | RPMI |
| 4 retina | RPE-1/hTert | 5.2 | DMEM/F12 | 4 gastric | NCC59 | 0.9 | RPMI |
| 5 lung | WI38 | 3.4 | DMEM | 5 gastric | SNU620 | 1.0 | RPMI |
| | | | | 6 gastric | SNU1 | 0.9 | RPMI |
| | | | | 7 gastric | AZ521 | 2.4 | RPMI |
| | | | | 8 gastric | SGCC-GC38 | 1.9 | RPMI |
| | | | | 9 gastric | SNU216 | 1.9 | RPMI |
| | | | | 10 gastric | SNU1750 | 0.7 | RPMI |
| | | | | 11 gastric | SNU-16 | 1.0 | RPMI |
| | | | | 12 gastric | SCH | 1.7 | RPMI |
| | | | | 13 gastric | MKN45 | 1.0 | DMEM |
| | | | | 14 gastric | AGS | 1.1 | RPMI |
| | | | | 15 gastric | IM95 | 1.0 | DMEM |
| | | | | 16 gastric | NUGC3 | 1.0 | RPMI |
| | | | | 17 gastric | NCC19 | 4.6 | RPMI |
| | | | | 18 colon | HCT15 | 0.5 | RPMI |
| | | | | 19 colon | DLD1 | 1.2 | RPMI |
| | | | | 20 colon | HT29 | 0.9 | RPMI |
| | | | | 21 colon | HCT116 | 1.8 | DMEM |
| | | | | 22 liver | Hep3 | 3.7 | DMEM |
| | | | | 23 liver | Huh7 | 1.2 | DMEM |
| | | | | 24 liver | SNU449 | 2.3 | RPMI |
| | | | | 25 breast | MDA-MB-231 | 1.1 | DMEM |
| | | | | 26 breast | MCF7 | 1.2 | DMEM |
| | | | | others | | | |
| | | | | 27 king | A549 | 2.0 | DMEM |
| | | | | 28 cervix | HeLa | 1.4 | DMEM |
| | | | | 29 bone | U2OS | 1.5 | DMEM |

A series of engineered human BJ cell lines (Voorhoeve, P. M. & Agami, R. Cancer Cell 4: 311-319 (2003)) were utilized to delineate the molecular basis for a131-mediated tumor cell-selectivity (FIG. 1F), and it was found that inhibition of the p16-pRb, p53 and PP2A tumor suppressor pathways in various combinations did not significantly contribute to a131-induced cell death. In contrast, 4-OHT-induced acute activation of H-RasV12-ER alone was sufficient to sensitize normal BJ cells to a131-induced cell death, and this effect was further enhanced in the context of transformed BJ cells (FIG. 1F). Together, these data indicate that a131 displays a strong selective lethality against Ras-activated or -transformed cells.

### Example 3: Normal Cells Undergo Reversible Cell Cycle Arrest

Consistent with a131-induced aneuploidy in transformed cells (FIG. 1C, panel d′), time-lapse analysis revealed that a131 treatment immediately induced mitotic arrest in transformed BJ cells (FIG. 3C) concomitant with massively misaligned chromosomes (FIG. 1G; FIG. 3D), which subsequently misaggregated into daughter cells often with catastrophic multipolar division, leading to cell death (not shown). In contrast, such catastrophic cell division rarely occurred in a131-treated normal BJ cells (not shown) and demonstrated significantly fewer mitotic defects than those in transformed cells (FIGS. 3C and 3E). Detailed analysis using high-resolution immunofluorescence microscopy revealed that a131 caused de-clustered centrosomes and multipolar mitotic-spindles in transformed BJ (FIGS. 1G and 1H) and other cancer cells (FIG. 3E), since most cancer cells contain supernumerary centrosomes clustered in a bipolar manner before division⁸. In contrast, functional bipolar spindle formed in a majority of a131-treated normal BJ cells (FIG. 1H; FIG. 3F) despite a slight decrease in metaphase spindle length (FIG. 3G). Together, these data suggest a131 as a potent antimitotic agent that preferentially kills cancer and transformed cells by inducing immediate cancer-selective mitotic catastrophe in vitro, while it arrests normal cells at the G₁/S phase of the cell cycle in a reversible manner, which explains a broad-spectrum of its anticancer effects (FIGS. 3A and 3B).

### Example 4: In Vivo Efficacy of Cancer-Selective Compounds

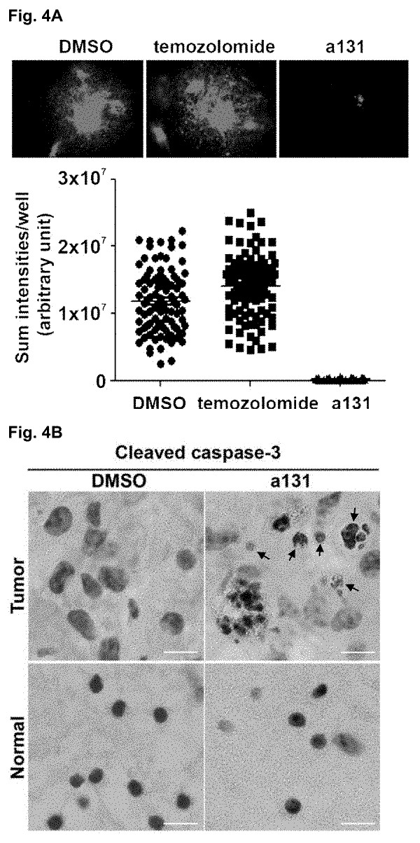

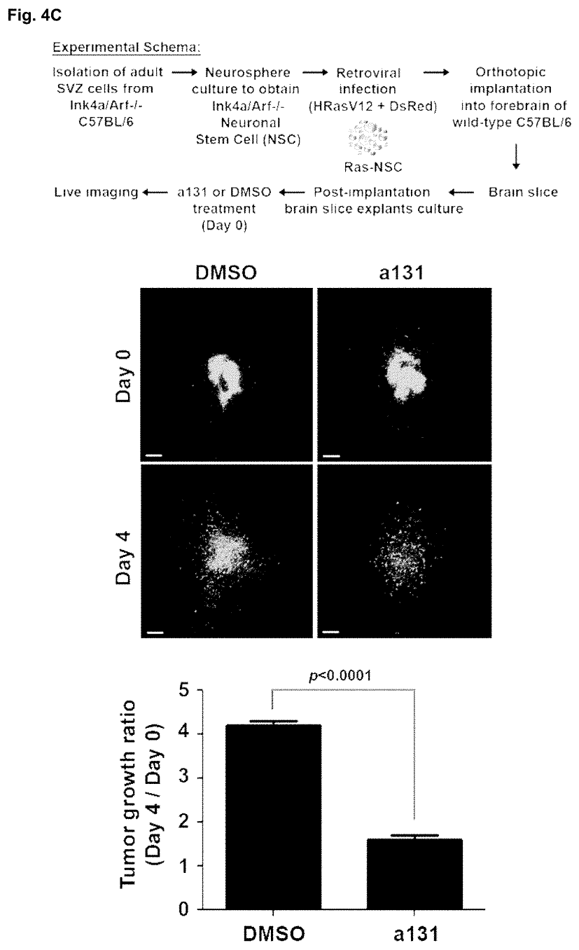

The antitumor activities of a131 and of b5, a derivative of a131 designed to improve aqueous solubility, were further determined in mouse xenograft models derived from both HCT-15 human colon adenocarcinoma cells and MDA-MB-231 human breast tumor cells harboring mutant K-RasG13D. As expected, paclitaxel did not show significant antitumor activity against HCT-15 (FIG. 1I), whereas, both oral and intraperitoneal injections of a131 and b5 demonstrated marked antitumor efficacies without any body weight loss (FIG. 1I) and cancer cell death as determined by TUNEL staining (FIG. 1J). As observed in in vitro tissue culture, a131 caused massively misaligned chromosomes with multipolar spindles in tumor sections (FIG. 1K). Moreover, in a tumor spheroid culture or orthotopically implanted ex vivo model, a131 treatment significantly suppressed growth of Ras-driven glioma initiating cells (GICs) (FIGS. 4A and 4C). In addition, a131 induced apoptosis only in tumors, but not surrounding normal tissues in ex vivo model (FIG. 4B). Taken together, a131 is a unique compound with a potent and broad anticancer efficacy by inducing cancer-selective mitotic catastrophe in vitro, ex vivo and in vivo.

### Example 5: Identification of Chemotherapeutic and Chemoprotective Compounds

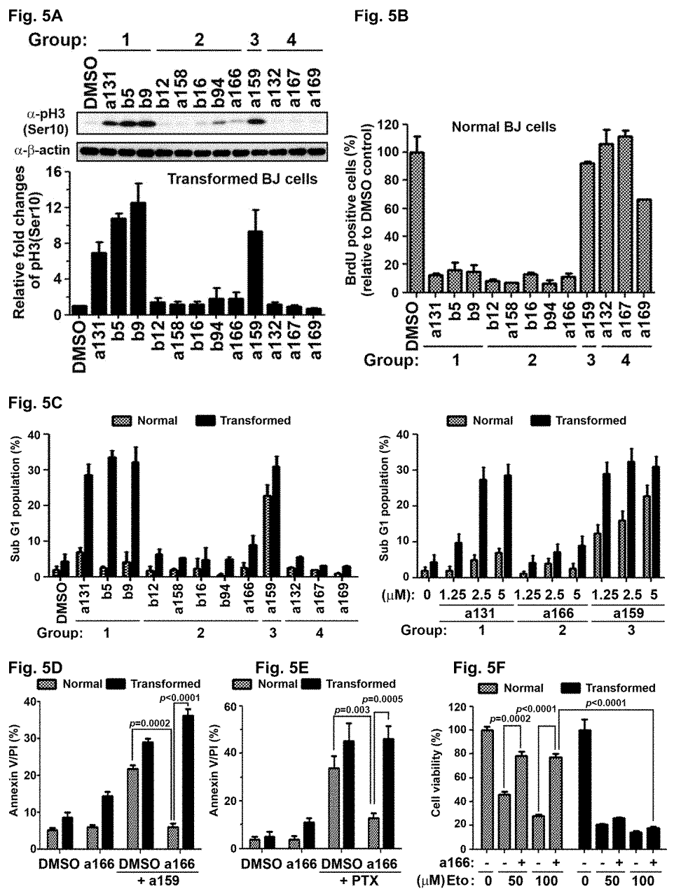

Using various derivatives of a131, it was found that the properties of a131 could be separated pharmacologically into two distinct pharmacophores (experimental details and a summary of the results are presented in FIGS. 5A-5C and Table 3). Through the analysis of a131 structure-and-activity relationship (SAR), these compounds were classified into four groups: Group 1 compounds, which possess the dual-inhibitory properties of both causing the arrest of normal BJ cells at the G₁/S phase and also causing mitotic arrest/catastrophe in transformed BJ cells (e.g. a131, b5); Group 2 compounds, which only cause the arrest of normal BJ cells at the G₁/S phase (e.g. a166); Group 3 compounds, which cause mitotic arrest/catastrophe in transformed BJ cells, but do not arrest normal BJ cells at the G₁/S phase (e.g. a159); and Group 4 compounds, which are inactive or weakly active (e.g. a132). Importantly, only Group 1 compounds retained the ability to selectively kill transformed BJ cells (FIG. 5C). In contrast, Group 2 and Group 4 compounds failed to kill either normal or transformed cell lines, while compounds in Group 3 killed both normal and transformed cell lines with much less selectivity than those in Group 1 (FIG. 5C). Notably, a131-like cancer-selective lethality was reproduced by combining compounds in Groups 2 and 3 (FIG. 5D). Moreover, while paclitaxel and etoposide treatment alone showed minimal selectivity against transformed BJ cells, pre-treatment with a166 in Group 2 markedly augmented such selectivity by protecting normal BJ cells from chemotherapeutic toxicity (FIGS. 5E and 5F). Together, these data suggest that the dual-inhibitory property of compounds in Group 1 (e.g. a131) is essential to achieve cancer-selective lethality. Furthermore, compounds in Group 2 (e.g. a166) and Group 3 (e.g. a159) can be classified as chemoprotective and chemotherapeutic agents, respectively.

TABLE 3
List of compounds with their structures clarified into 4 groups based
on a131 SAR analysis. Compounds in Group 1 are
both chemoprotective and selectively
chemotherapeutic, while compounds in Group 2 are chemoprotective.

| | |
|---|---|
| Name | Structure |

Group 1

| | |
|---|---|
| a131 | 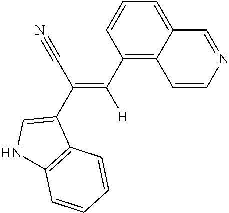 [SMILES: N#C/C(=C\c1cccc2cnccc12)c1c[nH]c2ccccc12] |
| b5 | 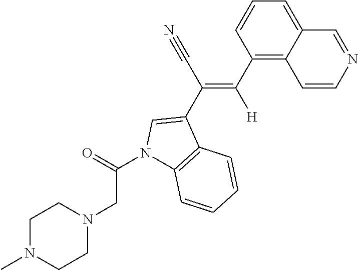 [SMILES: CN1CCN(CC(=O)n2cc(/C(C#N)=C/c3cccc4cnccc34)c3ccccc32)CC1] |
| b9 | 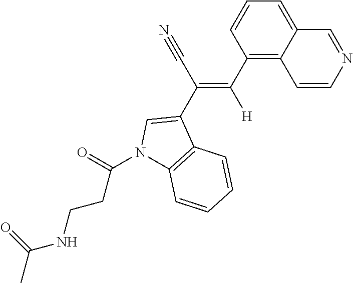 [SMILES: CC(=O)NCCC(=O)n1cc(/C(C#N)=C/c2cccc3cnccc23)c2ccccc21] |

Group 2

| | |
|---|---|
| b12 | 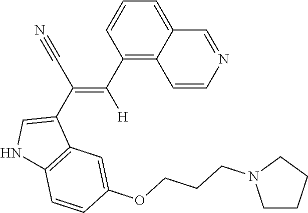 [SMILES: N#C/C(=C\c1cccc2cnccc12)c1c[nH]c2ccc(OCCCN3CCCC3)cc12] |
| a158 | 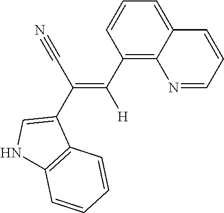 [SMILES: N#C/C(=C\c1cccc2cccnc12)c1c[nH]c2ccccc12] |
| b16 | 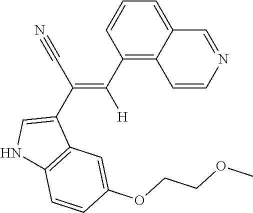 [SMILES: COCCOc1ccc2[nH]cc(/C(C#N)=C/c3cccc4cnccc34)c2c1] |
| b94 | 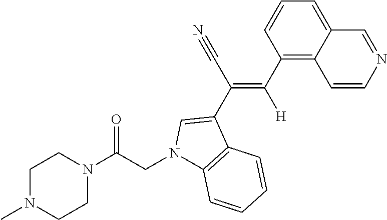 [SMILES: CN1CCN(C(=O)Cn2cc(/C(C#N)=C/c3cccc4cnccc34)c3ccccc32)CC1] |
| a166 | 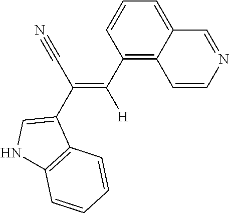 [SMILES: N#C/C(=C\c1cccc2cnccc12)c1c[nH]c2ccccc12] |

Group 3

| | |
|---|---|
| a159 | 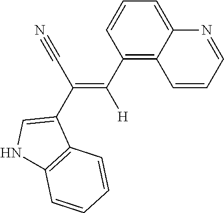 [SMILES: N#C/C(=C\c1cccc2ncccc12)c1c[nH]c2ccccc12] |

Group 4

| | |
|---|---|
| a132 | 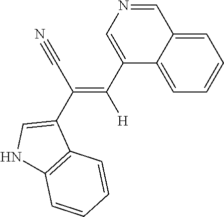 [SMILES: N#C/C(=C\c1cncc2ccccc12)c1c[nH]c2ccccc12] |
| a167 | 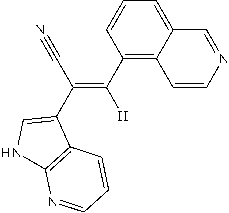 [SMILES: N#C/C(=C\c1cccc2cnccc12)c1c[nH]c2ncccc12] |
| a169 | 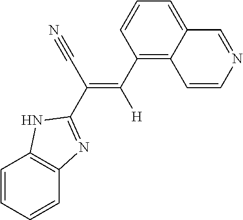 [SMILES: N#C/C(=C\c1cccc2cnccc12)c1nc2ccccc2[nH]1] |

### Example 6: PI5P4Ks Identified as Target for Inducing Growth Arrest

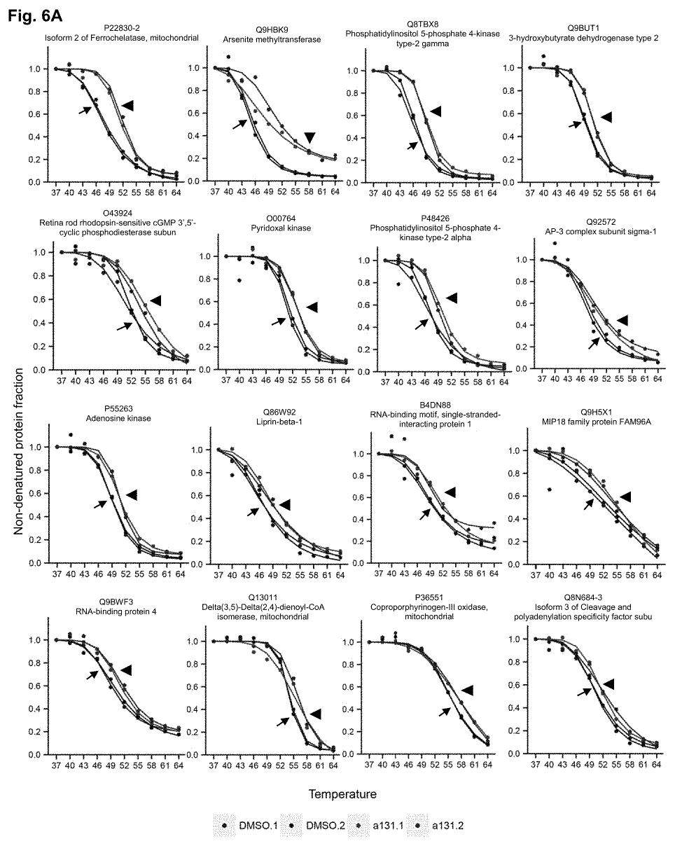

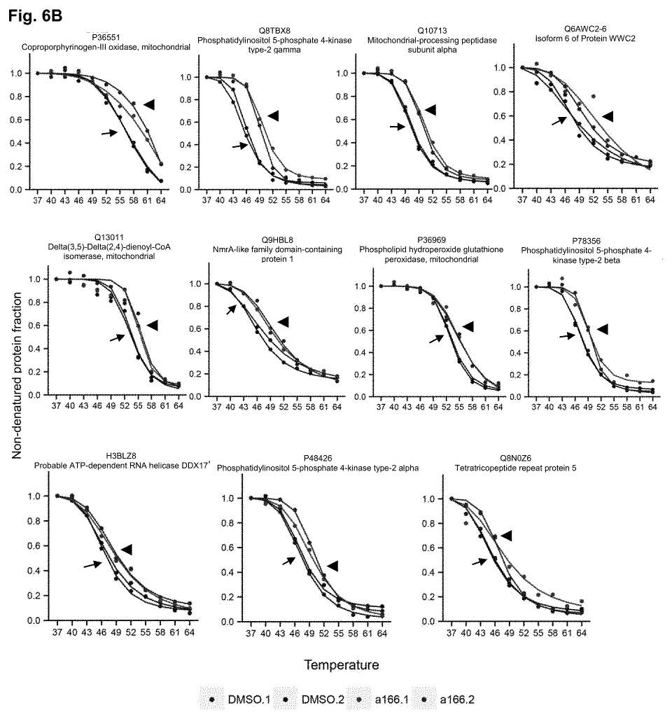

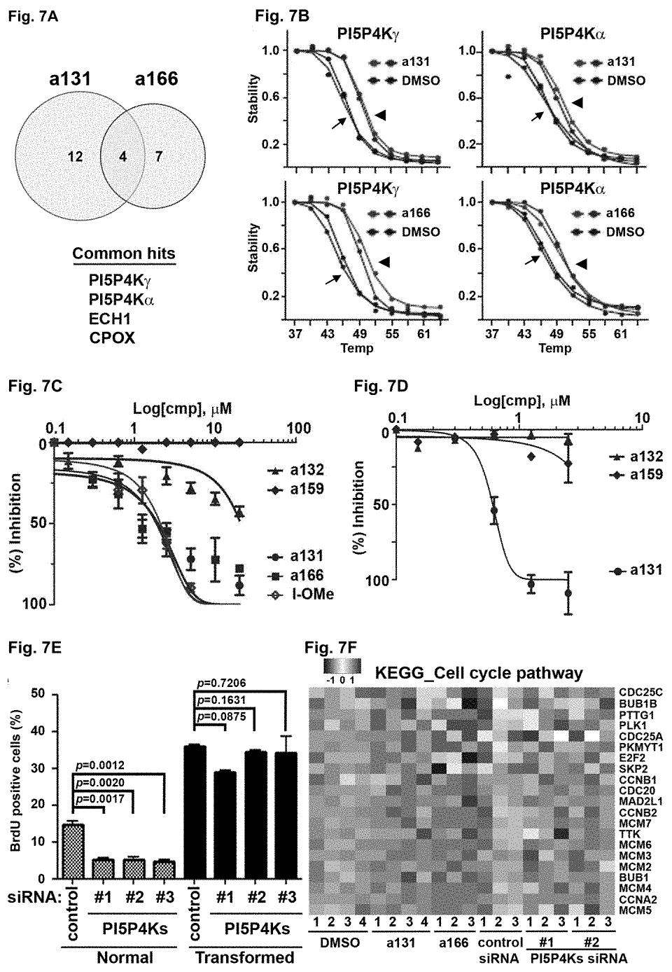

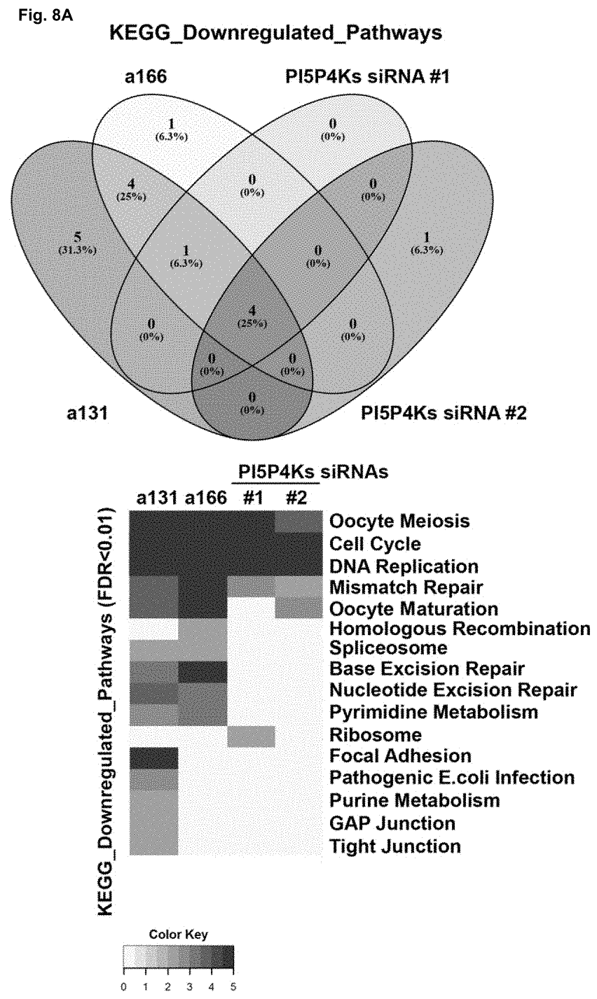

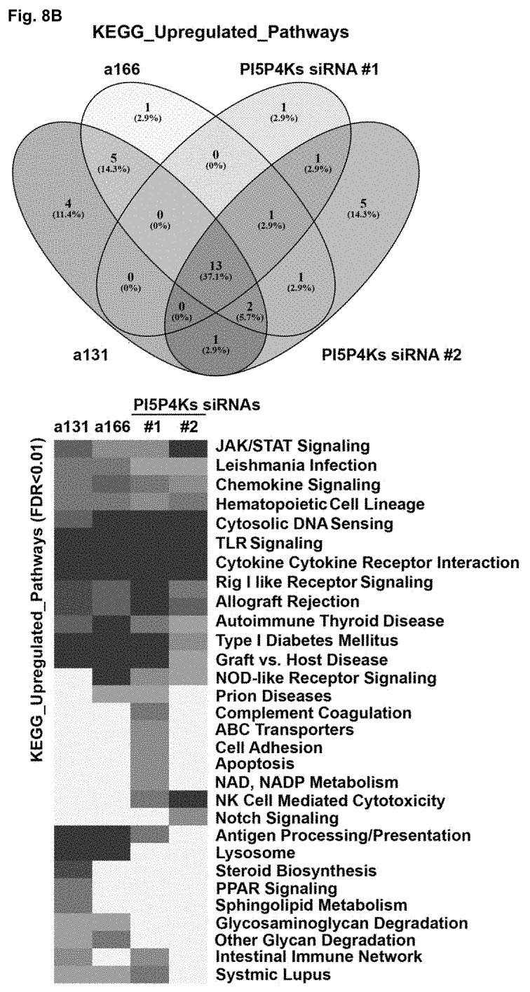

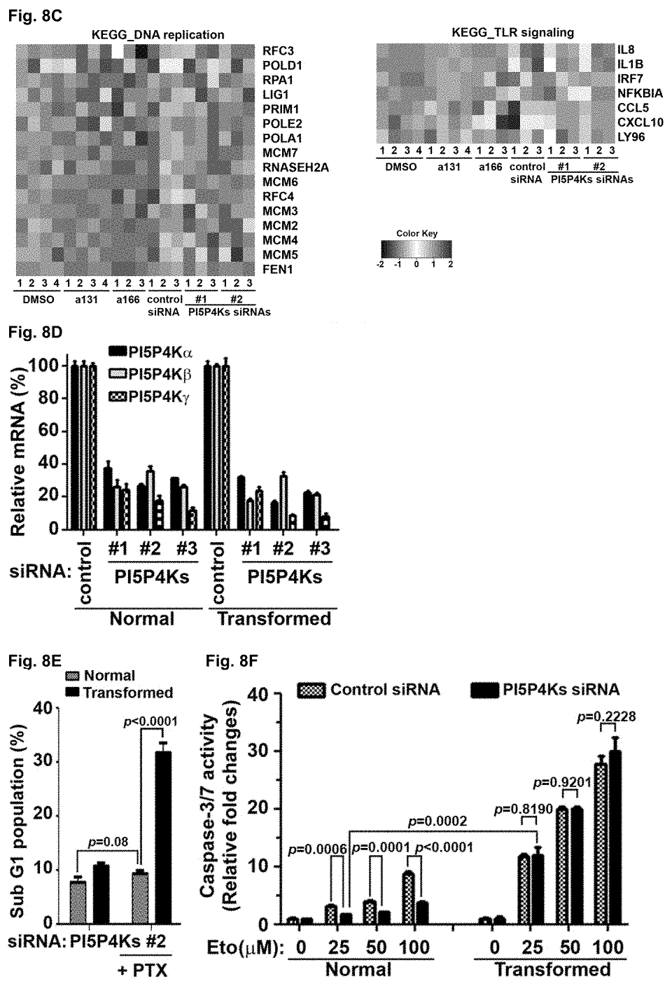

To identify cellular targets and signaling pathways of a131 that are responsible for arresting only normal BJ cells at the G₁/S phase of the cell cycle, the mass-spectrometry implementation of the cellular thermal shift assay (MS-CETSA) for target identification at the proteome level was explored. To increase confidence in target identification, both a131 and a166 were applied for CETSA analysis to find common target proteins. After collecting data covering >8,000 proteins in lysates of normal BJ cells, >4,000 proteins were used for each compound in the final analyses. Using ranking based on Euclidian distances and thermal shift size, 16 and 11 proteins were selected as potential significant hits for a131 and a166, respectively (FIGS. 6A-6B; Table 4). Ferrochelatase in a131 and coproporphyrinogen-III oxidase (CPOX) in a166 were identified as prominent hits. These two proteins of the heme synthesis pathway, however, have previously been identified as promiscuous binders of multiple drugs (Savitski, M. M. et al., Science 346: 55 (2014); Klaeger, S. et al., ACS Chem. Biol. 11: 1245-1254 (2016)), indicating their inhibition is unlikely to give the observed phenotypes of a131 and a166. Instead, the members of PI5P4Ks (phosphatidylinositol 5-phosphate 4-kinases)(Bulley, S. J., Clarke, J. H., Droubi, A., Giudici, M.-L. & Irvine, R. Adv. Biol. Regul. 57: 193-202 (2015); Clarke, J. H. & Irvine, R. F. Adv. Biol. Regul. 52: 40-45 (2012)) stood out as the most prominent common hits, which could constitute candidates for the pharmacological targets; two out of the three family members (PI5P4Kα and -γ) in a131 and all three family members (PI5P4Kα, β and γ) in a166 were identified as CETSA hits (FIGS. 7A and 7B; Table 4). Indeed, a131 was able to inhibit the kinase activity of purified PI5P4Kα in vitro as well as combined cellular PI5P4Ks with IC₅₀ of 1.9 μM and 0.6 μM, respectively (FIGS. 7C and 7D). Likewise, both a166 and I-OMe-AG-538, previously reported to show PI5P4Kα inhibition (Davis, M. I. et al., PloS one 8: e54127 (2013)), also inhibited the PI5P4Kα activity with IC₅₀ of 1.8 μM and 2.1 μM, respectively (FIG. 7C). Knockdown of all PI5P4K isoforms using three different sets of siRNAs (FIG. 8D) induced growth arrest only in normal BJ cells (FIG. 7E), a phenocopy of a131 and a166 treatment (FIG. 1C; FIGS. 5A and 5B). Moreover, GSEA and KEGG pathway analysis revealed that PI5P4Ks knockdown in normal BJ cells down-regulated the set of genes promoting cell cycle (FIG. 7f) with a significant number of comparably up- or down-regulated common cellular pathways as similar to a131 and a166 treatment (FIGS. 8A-8C). Similar to a166 treatment (FIGS. 5D-5F), PI5P4Ks knockdown also showed significant chemoprotective effects only in normal BJ cells from paclitaxel and etoposide treatment (FIGS. 8E and 8F). Together, these data suggest that PI5P4Ks are the cellular targets of a131 and a166. This is to the best of our knowledge the first study where MS-CETSA has been used to unravel pharmaceutical targets for hits from a phenotypic screen.

TABLE 4
List of CETSA hits that passed the selection criteria for a131 and a166 datasets.

| | | | |
|---|---|---|---|
| Uniprot id | Description | ED score | mean Delta Tm |

a131 hits

| | | | |
|---|---|---|---|
| P22830-2 | Isoform 2 of Ferrochelatase, mitochondrial | 0.8365653 | 3.81452563 |
| Q9HBK9 | Arsenite methyltransferase | 0.6214127 | 4.88431916 |
| Q8TBX8 | Phosphatidylinositol 5-phosphate 4-kinase type-2 gamma | 0.5988635 | 2.88860687 |
| Q9BUT1 | 3-hydroxybutyrate dehydrogenase type 2 | 0.5139135 | 1.82669061 |
| O43924 | Retinal rod rhodopsin-sensitive cGMP 3′,5′-cyclic phosphodiesterase subunit delta | 0.3091578 | 3.02401165 |
| O00764 | Pyridoxal kinase | 0.448649 | 2.01117597 |
| P48426 | Phosphatidylinositol 5-phosphate 4-kinase type-2 alpha | 0.3512946 | 2.36666224 |
| Q92572 | AP-3 complex subunit sigma-1 | 0.3486217 | 2.26435048 |
| P55263 | Adenosine kinase | 0.4157529 | 1.73616347 |
| Q86W92 | Liprin-beta-1 | 0.3386855 | 2.06564997 |
| B4DN88 | RNA-binding motif, single-stranded-interacting protein 1 | 0.3143895 | 2.01480829 |
| Q9H5X1 | MIP18 family protein FAM96A | 0.2909711 | 2.16601092 |
| Q9BWF3 | RNA-binding protein 4 | 0.3322612 | 1.88050949 |
| Q13011 | Delta(3,5)-Delta(2,4)-dienoyl-CoA isomerase, mitochondrial | 0.3540918 | 1.51480598 |
| P36551 | Coproporphyrinogen-III oxidase, mitochondrial | 0.3274074 | 1.61311962 |
| Q8N684-3 | Isoform 3 of Cleavage and polyadenylation specificity factor subunit 7 | 0.3008003 | 1.65314476 |

a166 hits

| | | | |
|---|---|---|---|
| P36551 | Coproporphyrinogen-III oxidase, mitochondrial | 0.5714591 | 4.0795771 |
| Q8TBX8 | Phosphatidylinositol 5-phosphate 4-kinase type-2 gamma | 0.5295564 | 3.7764899 |
| Q10713 | Mitochondrial-processing peptidase subunit alpha | 0.618153 | 2.7225026 |
| Q6AWC2-6 | Isoform 6 of Protein WWC2 | 0.3773994 | 3.4729223 |
| Q13011 | Delta(3,5)-Delta(2,4)-dienoyl-CoA isomerase, mitochondrial | 0.5429673 | 2.0978877 |
| Q9HBL8 | NmrA-like family domain-containing protein 1 | 0.3484292 | 2.6746446 |
| P36969 | Phospholipid hydroperoxide glutathione peroxidase, mitochondrial | 0.456269 | 1.9308683 |
| P78356 | Phosphatidylinositol 5-phosphate 4-kinase type-2 beta | 0.4050132 | 2.0022629 |
| H3BLZ8 | Probable ATP-dependent RNA helicase DDX17 | 0.3453529 | 2.0127912 |
| P48426 | Phosphatidylinositol 5-phosphate 4-kinase type-2 alpha | 0.3024599 | 2.2482015 |
| Q8N0Z6 | Tetratricopeptide repeat protein 5 | 0.2801698 | 2.1055021 |

Details of the proteins which were scored as hits from the a131 and the a166 datasets are shown. The uniprot ids of the hits, protein names, Euclidean Distance (ED) scores and the mean ΔTm are shown. The ED score of the thermal shifts of the proteins were calculated and hit lists were generated using a cutoff at median + 2.75*MAD (Median Absolute Deviation). ΔTm value of thermal shifts were calculated as average deviations between control and treated samples at 0.5 fold change, and proteins with significant positive ΔTm value of median + 2.75*MAD were selected. The proteins with both significant ED score and significant ΔTm value were selected as the final hit lists corresponding to 16 and 11 proteins for a131 and a166, respectively. The hit lists were then ranked in descending ED score and ΔTm value.

### Example 7: Chemoprotective Compounds Act by Modulating PI3K-Interacting Protein1

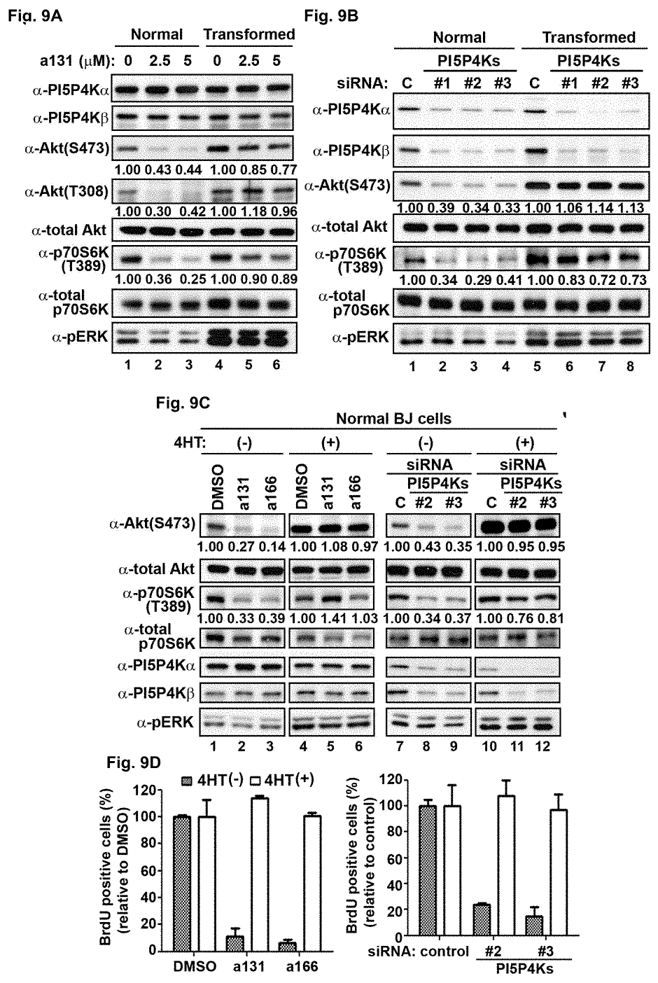

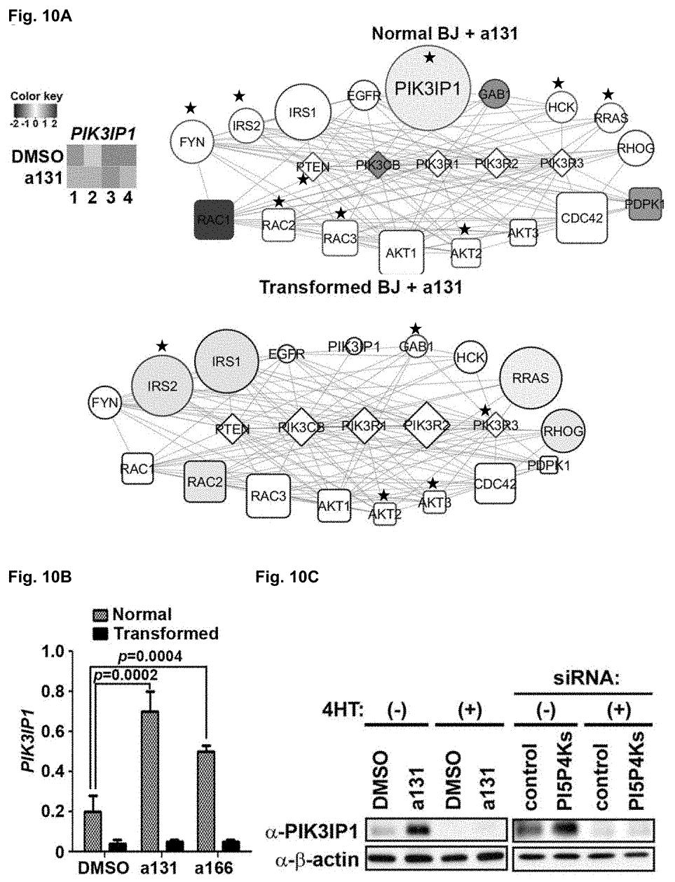

PI5P4K loss-of-function mutants in Drosophila possessing only one isoform of PI5P4K show inhibition of the PI3K/Akt/mTOR pathway (Gupta, A. et al., Proc. Natl. Acad. Sci. U.S.A 110: 5963-5968 (2013)). Importantly, a131 treatment or PI5P4Ks knockdown using three different sets of siRNAs also consistently caused inhibition of the PI3K/Akt/mTOR pathway only in normal BJ cells, but not in transformed counterparts (FIGS. 9A and 9B). Likewise, 4-OHT-induced H-RasV12-ER activation was sufficient to reactivate the PI3K/Akt/mTOR pathway in normal BJ cells even after a131 and a166 treatment or PI5P4Ks knockdown (FIG. 9C), which correlates with activated Ras overriding a131-, a166- or PI5P4Ks knockdown-induced growth arrest in normal BJ cells (FIG. 9D). Together, these data suggest a role of PI5P4Ks in promoting the PI3K/Akt/mTOR signaling pathway in a Ras-dependent manner.

The molecular components that control the interactions between the Ras/Raf/MEK/ERK and the PI3K/Akt/mTOR pathways are not fully understood. Neither a131 and a166 treatment nor PI5P4Ks knockdown inhibited the Ras/Raf/MEK/ERK pathway in normal BJ cells, as determined by ERK phosphorylation (FIGS. 9A-9C). Thus, to determine how a131 controlled the PI3K/Akt/mTOR pathway in a Ras-dependent manner, the inventors interrogated differences in the gene expression levels of known regulators and effectors associated with PI3K in normal and transformed BJ cells upon a131 treatment. Strikingly, among these genes, the PI3K-interacting protein 1 gene (PIK3IP1) was significantly up-regulated only in a131-treated normal BJ cells (FIG. 10A). Indeed, qRT-PCR and immunoblot analysis confirmed up-regulation of PIK3IP1 at both the mRNA and protein levels in either a131- and a166-treated or PI5P4Ks knockdown normal BJ cells (FIGS. 10B and 10C). Conversely, PIK3IP1 mRNA expression in transformed BJ cells was not only significantly lower, but also was unresponsive to a131 and a166 treatment (FIG. 10B). Moreover, 4-OHT-induced H-RasV12-ER activation was sufficient to down-regulate mRNA and protein levels of PIK3IP1 in a131-treated normal BJ cells (FIG. 100) and to dissociate RNA polymerase II (Pol II) from the PIK3IP1 promoter (FIG. 10D). In contrast, pharmacological inhibition of MEK attenuated H-RasV12-ER-induced PIK3IP1 suppression (FIG. 10E), suggested the molecular basis for positive cross-talk between the Ras/Raf/MEK/ERK and PI3K/Akt/mTOR pathways is mediated by negative transcriptional regulation of PIK3IP1.

PIK3IP1 binds the p110 catalytic subunit of PI3K heterodimers and inhibits PI3K catalytic activity, which leads to inhibition of the PI3K/Akt/mTOR pathway, and PIK3IP1 dysregulation contributes to carcinogenesis (Bitler, B. G. et al., Nat. Med. 21: 231-238 (2015); He, X. et al., Cancer Res. 68: 5591-5598 (2008); Zhu, Z. et al., Biochem. Biophys. Res. Commun. 358: 66-72 (2007); Wong, C. C. et al., Nat. Genet. 46: 33-38 (2014)). Therefore, it was determined whether a131-mediated up-regulation of PIK3IP1 was indeed responsible for the observed inhibition of the PI3K/Akt/mTOR pathway and the G₁/S phase transition in normal BJ cells. Indeed, PIK3IP1 knockdown in normal BJ cells significantly restored activation of the PI3K/Akt/mTOR pathway and rescued the population of BrdU-positive proliferative cells, which were suppressed by a131 treatment (FIG. 10F). Together, these data reveal positive cross-talk between the Ras and PI3K pathways via the Ras-IPIK3IP1-IPI3K signaling network.

Of therapeutic importance is the observation that PI5P4Ks inhibition by a131 and a166 caused reversible growth arrest in normal cells by transcriptionally upregulating PIK3IP1, a suppressor of the PI3K/Akt/mTOR pathway (Bitler, B. G. et al., Nat. Med. 21: 231-238 (2015); He, X. et al., Cancer Res. 68: 5591-5598 (2008); Zhu, Z. et al., Biochem. Biophys. Res. Commun. 358: 66-72 (2007); Wong, C. C. et al., Nat. Genet. 46: 33-38 (2014)).

### Example 8: Selective Killing Effects of a131 in Various Human Cancer Cells, but not Normal Cells

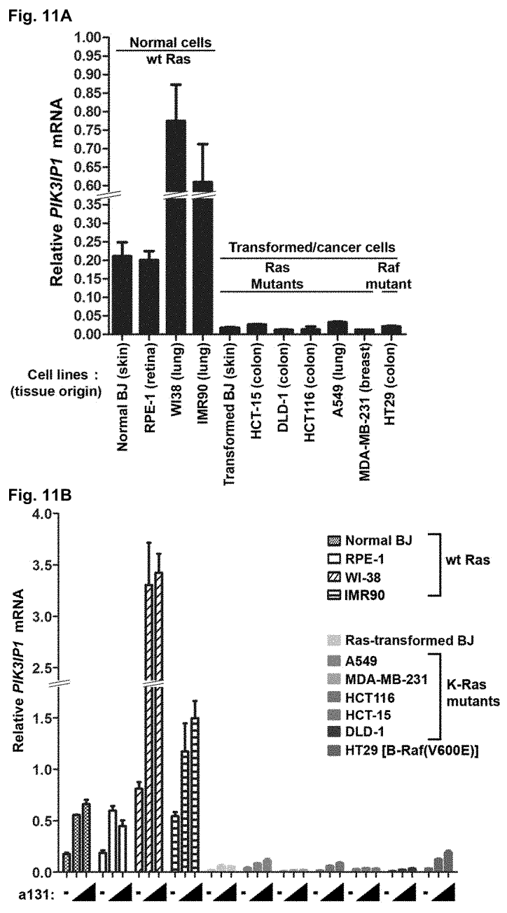

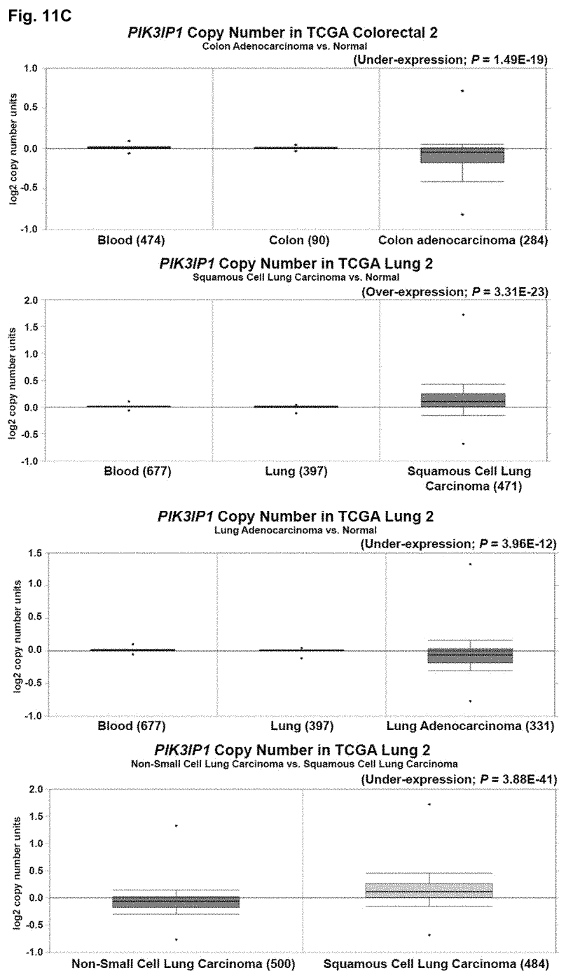

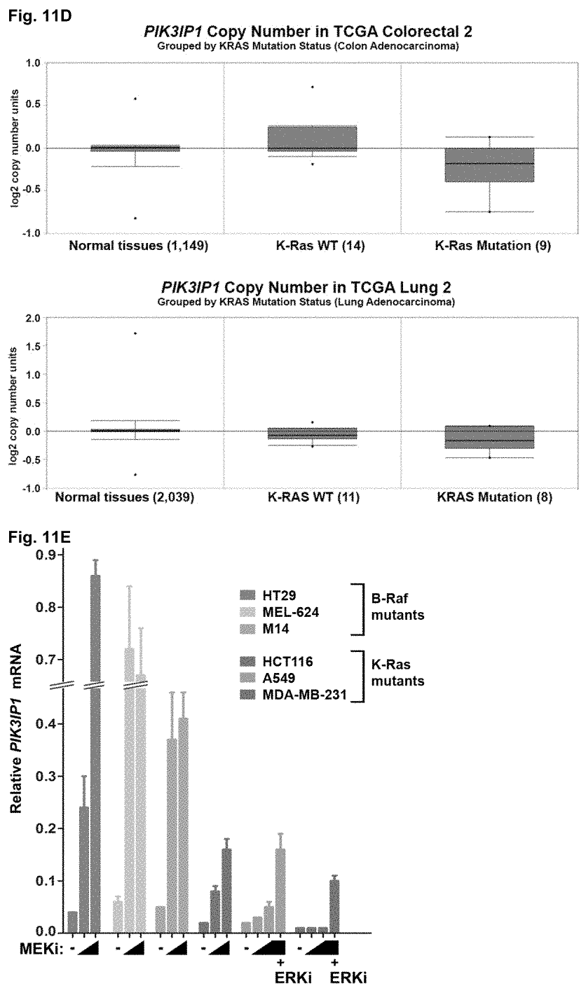

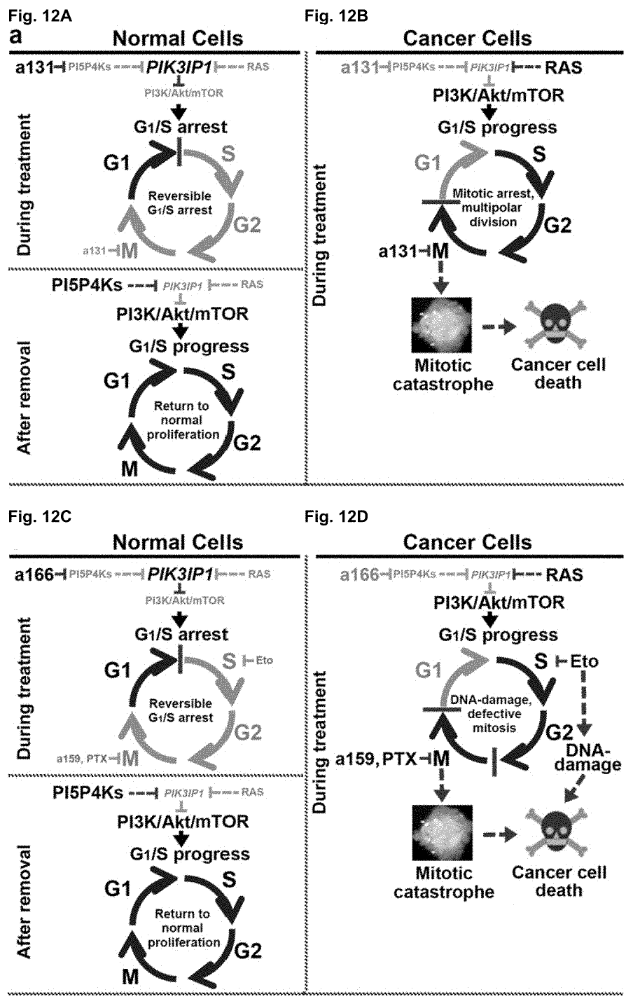

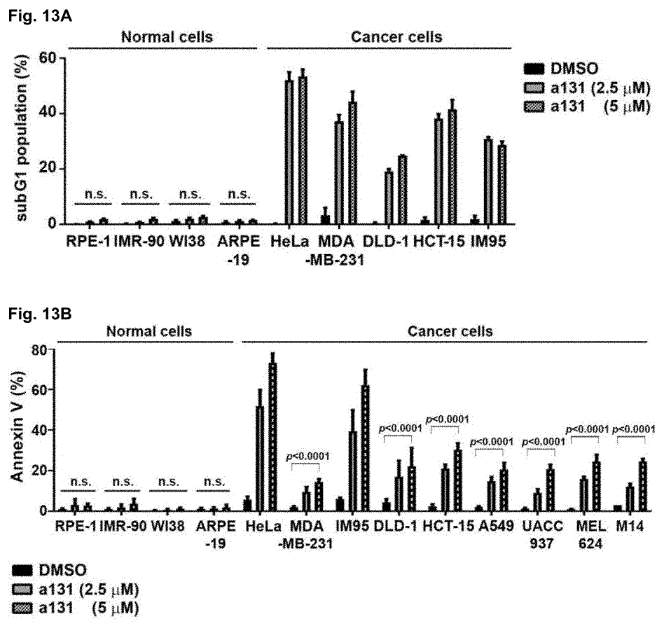

PIK3IP1 mRNA levels were not only considerably lower in Ras- and Raf-mutant cancer cells compared with normal cells (FIG. 11A), but a131- and a166-mediated induction of PIK3IP1 was also significantly attenuated in these cancer cells, unlike normal cells (FIG. 10G; FIG. 11B). Similarly, analysis of an Oncomine dataset derived from patient samples revealed that PIK3IP1 expression was significantly lower in human colorectal and lung adenocarcinomas, where Ras mutations and activation of Ras signaling pathways are common, compared with their corresponding normal tissues or squamous cell lung carcinoma where Ras mutations are uncommon (FIG. 11C). Indeed, a negative correlation between PIK3IP1 expression and Ras mutation status in human colorectal and lung adenocarcinomas was observed (FIG. 11D). Conversely, pharmacological inhibition of MEK and ERK significantly increased PIK3IP1 expression in many Ras- and Raf-mutant cancer cells (FIG. 10H; FIG. 11E), while this observed de-repression of PIK3IP1 was more prominent in most of Raf-mutant cancer cells (FIG. 11E), indicating that high MAPK activity is responsible for the suppression of PIK3IP1. Furthermore, this de-repression of PIK3IP1 correlated with concomitant inhibition of the PI3K/Akt/mTOR pathway in HCT116, A549 and transformed BJ cells, but not in those cells unable to de-repress PIK3IP1 (FIG. 10H). Conversely, PIK3IP1 knockdown significantly restored activation of the PI3K/Akt/mTOR pathway and suppressed cell death induced by inhibition of MEK and ERK (FIG. 10I), further indicating positive cross-talk between the Ras and PI3K pathways via the Ras-IPIK3IP1-1PI3K signaling network for cancer cell proliferation and survival.

FIG. 13 provides additional support to show that a131 is selective towards cancer cells, and treated cancer cells undergo cell death via apoptosis, but not other forms of cell death. The percentage of subG1 population and cells expressing Annexin V is significantly higher (p<0.0001) for cancer cells treated with a131 (at both 2.5 μM and 5 μM dosages) compared to cancer cells treated with DMSO.

### REFERENCES

Any listing or discussion of an apparently prior-published document in this specification should not necessarily be taken as an acknowledgement that such document is part of the state of the art or is common general knowledge.
Agrawal N, Dasaradhi P V N, Mohmmed A, Malhotra P, Bhatnagar R K, Mukherjee S K. RNA Interference: Biology, Mechanism, and Applications. Microbiology and Molecular Biology Reviews. 67(4): 657-685 (2003).
Angart P. A. et al., Terminal Duplex Stability and Nucleotide Identity Differentially Control siRNA Loading and Activity in RNA Interference. Nucleic Acid Therapeutics. 26(5), 309-317 (2016).
Bitler, B. G. et al. Synthetic lethality by targeting EZH2 methyltransferase activity in ARID1A-mutated cancers. Nat. Med. 21, 231-238 (2015).
Bulley, S. J., Clarke, J. H., Droubi, A., Giudici, M.-L. & Irvine, R. F. Exploring phosphatidylinositol 5-phosphate 4-kinase function. Adv. Biol. Regul. 57, 193-202 (2015).
Clarke, J. H. & Irvine, R. F. The activity, evolution and association of phosphatidylinositol 5-phosphate 4-kinases. Adv. Biol. Regul. 52, 40-45 (2012).
Core_Team, R. R: A language and environment for statistical computing. R Foundation for Statistical Computing, Vienna, Austria. (2014).
Davis, M. I. et al. A Homogeneous, High-Throughput Assay for Phosphatidylinositol 5-Phosphate 4-Kinase with a Novel, Rapid Substrate Preparation. PloS one 8, e54127, (2013).
Gupta, A. et al. Phosphatidylinositol 5-phosphate 4-kinase (PIP4K) regulates TOR signaling and cell growth during Drosophila development. Proc. Natl. Acad. Sci. U.S.A 110, 5963-5968 (2013).
He, X. et al. PIK3IP1, a Negative Regulator of PI3K, Suppresses the Development of Hepatocellular Carcinoma. Cancer Res. 68, 5591-5598 (2008).
Jokinen, E. & Koivunen, J. P. MEK and PI3K inhibition in solid tumors: rationale and evidence to date. Ther. Adv. Med. Oncol. 7, 170-180 (2015).
Kanehisa, M. & Goto, S. KEGG: Kyoto Encyclopedia of Genes and Genomes. Nucl. Acids Res. 28, 27-30, (2000).
Klaeger, S. et al. Chemical Proteomics Reveals Ferrochelatase as a Common Off-target of Kinase Inhibitors. ACS Chem. Biol. 11, 1245-1254 (2016).
Langer R., New methods of drug delivery. Science 249: 1527-33 (1990)
Mayer, I. A. & Arteaga, C. L. Targeting PI3K signalling in cancer: opportunities, challenges and limitations. Annu. Rev. Med. 67, 11-28 (2016).
McCormick, F. KRAS as a Therapeutic Target. Clin. Cancer Res. 21, 1797-1801 (2015).
Remington The Science and Practice of Pharmacy, 19th ed., Mack Printing Company, Easton, Pa. (1995)
Saga, I. et al. Integrated analysis identifies different metabolic signatures for tumor-initiating cells in a murine glioblastoma model. Neuro. Oncol. 16, 1048-1056, (2014).
Sampetrean, O. et al. Invasion Precedes Tumor Mass Formation in a Malignant Brain Tumor Model of Genetically Modified Neural Stem Cells. Neoplasia 13, 784-791 (2011)
Savitski, M. M. et al. Tracking cancer drugs in living cells by thermal profiling of the proteome. Science 346, 55 (2014).
Subramanian, A. et al. Gene set enrichment analysis: A knowledge-based approach for interpreting genome-wide expression profiles. Proc. Natl. Acad. Sci. U.S.A 102, 15545-15550, (2005).
Voorhoeve, P. M. & Agami, R. The tumor-suppressive functions of the human INK4A locus. Cancer Cell 4, 311-319 (2003).
Ward, J. H. Hierarchical Grouping to Optimize an Objective Function. Journal of the American Statistical Association 58, 236-244, (1963).
Wong, C. C. et al. Inactivating CUX1 mutations promote tumorigenesis. Nat. Genet. 46, 33-38 (2014).
Zhu, Z. et al. PI3K is negatively regulated by PIK3IP1, a novel p110 interacting protein. Biochem. Biophys. Res. Commun. 358, 66-72 (2007).

1-31. (canceled)

32. An in vitro or in vivo method for modulating cell survival, comprising contacting at least one cell with at least one phosphatidylinositol 5-phosphate 4-kinase family (PI5P4Ks) modulator and/or at least one phosphoinositide 3-kinase-interacting protein 1 (PIK3IP1) modulator.

33. The method according to claim 32, wherein said at least one modulator
i) inhibits PI5P4Kα, PI5P4Kδ and/or PI5P4Kγ activity, or
ii) inhibits PI5P4Kα, PI5P4Kδ and PI5P4Kγ activity,
and/or activates PIK3IP1 to cause cell cycle arrest at G1/S in normal cells, but not in transformed or hyperproliferative cells.

34. The method of claim 32, wherein, if said at least one modulator inhibits PI5P4K activity and causes cell cycle arrest at G1/S in normal cells, the modulator is chemoprotective for said normal cells.

35. The method of claim 34, wherein, if said at least one modulator inhibits PI5P4K activity and causes cell cycle arrest at G1/S in normal cells but not in transformed or hyperproliferative cells, the modulator is chemoprotective for said normal cells.

36. The method of claim 32, wherein the modulator is;
(a) at least one compound selected from group 1 and/or group 2 in Table 3, or a variant thereof;
(b) at least one siRNA that inhibits PI5P4Kα, PI5P4Kδ and/or PI5P4Kγ activity; or
(c) at least one MEK or ERK inhibitor that activates PIK3IP1.

37. The method of claim 36, wherein the at least one siRNA is directed to a PI5P4Kα, PI5P4Kδ and/or PI5P4Kγ nucleic acid sequence selected from the group consisting of SEQ ID NO: 19, 21, 23, 25, 27, 29 and 31.

38. A composition comprising at least one modulator that inhibits PI5P4Kα, PI5P4Kδ and/or PI5P4Kγ activity and/or activates PIK3IP1 to cause cell cycle arrest at G1/S in normal cells, but not in transformed or hyperproliferative cells for use in chemoprotection of normal cells and/or chemotherapy of transformed or hyperproliferating cells.

39. The composition according to claim 38, wherein the at least one modulator is a Group 1 or Group 2 compound in Table 3 or variant thereof, at least one siRNA that inhibits PI5P4Kα, PI5P4Kβ and/or PI5P4Kγ activity and/or a MEK or ERK inhibitor that activates PIK3IP1.

40. The composition according to claim 38, wherein the at least one siRNA is directed to a PI5P4Kα, PI5P4Kβ and/or PI5P4Kγ having a nucleic acid sequence selected from the group consisting of SEQ ID NO: 19, 21, 23, 25, 27, 29 and 31.

41. A method for identifying compounds that modulate PI5P4Ks activity and are suitable for use in treating a hyperproliferative disorder or disease, said method comprising:
(a) providing at least one cell comprising said PI5P4Ks;
(b) contacting said at least one cell with at least one test compound;
(c) detecting whether PI5P4Kα, PI5P4Kβ and/or PI5P4Kγ activity is inhibited and the at least one cell enters cell cycle arrest at G1/S phase, and comparing with untreated cells.

42. The method of claim 41, wherein, if the at least one test compound inhibits PI5P4Kα, PI5P4Kβ and/or PI5P4Kγ activity and causes cell cycle arrest at G1/S phase in normal cells, said at least one test compound is chemoprotective for normal cells during chemotherapy.

43. The method of claim 42, wherein inhibition of PI5P4Kα, PI5P4Kβ and/or PI5P4Kγ activity up-regulates PIK3IP1 and inhibits a PI3K/Akt/mTOR pathway in said normal cells.

44. The method of claim 42, wherein Ras activation in transformed or hyperproliferative cells suppresses PIK3IP1 expression and its up-regulation by PI5P4Ks, thereby counteracting PI5P4Ks-induced inhibition of the PI3K/Akt/mTOR pathway in said transformed or hyperproliferative cells.

45. The method of claim 44, wherein said transformed or hyperproliferative cells are Ras-activated cancer cells.

1546. The method of claim 41, wherein the inhibition of PI5P4K activity is indicated by an up-regulation of PIK3IP1 at both the mRNA and protein levels compared to untreated cells.

47. A method of treatment of a hyperproliferative disease or disorder, which method comprises the administration of an effective amount of at least one compound which inhibits PI5P4Kα, PI5P4Kβ and/or PI5P4Kγ activity and causes cell cycle arrest at G1/S phase in normal cells but not in transformed or hyperproliferating cells and, optionally, an effective amount of an anti-hyperproliferation agent.

48. The method of claim 47, wherein said transformed or hyperproliferating cells are Ras-activated cancer cells.

49. The method of claim 47, wherein said anti-hyperproliferation agent is a chemotherapeutic agent.

50. The method of claim 47, wherein said at least one compound is a small molecule, aptamer or siRNA.

51. The method of claim 47, wherein said at least one compound is selected from the Group 2 compounds in Table 3, or a variant thereof.

52. The method of claim 47, wherein said at least one compound also causes transformed or hyperproliferating cells to undergo mitotic catastrophe and is selected from the Group 1 compounds in Table 3, or a variant thereof.


SEQUENCE LISTING (32 sequences)

SEQ ID NO: 1 - DNA, 21 bp, Artificial Sequence
  siRNA target human PI5P4K alpha, pool #1
ctgcccgatggtcttccgtaa

SEQ ID NO: 2 - DNA, 21 bp, Artificial Sequence
  siRNA target human PI5P4K beta, pool #1
cacgatcaatgagctgagcaa

SEQ ID NO: 3 - DNA, 21 bp, Artificial Sequence
  siRNA target human PI5P4K gamma, pool #1
ccggagcagtatgctaagcga

SEQ ID NO: 4 - DNA, 21 bp, Artificial Sequence
  siRNA target human PI5P4K alpha, pool #2
cggcttaatgttgatggagtt

SEQ ID NO: 5 - DNA, 21 bp, Artificial Sequence
  siRNA target human PI5P4K beta, pool #2
ccctcgatctatttccttctt

SEQ ID NO: 6 - DNA, 21 bp, Artificial Sequence
  siRNA target human PI5P4K alpha, pool #3
cctcggacagacatgaacatt

SEQ ID NO: 7 - DNA, 21 bp, Artificial Sequence
  siRNA target human PI5P4K beta, pool #3
caaacgcttcaacgagtttat

SEQ ID NO: 8 - DNA, 21 bp, Artificial Sequence
  siRNA target human PI5P4K gamma, pool #3
ccgagtcagtgtggacaacga

SEQ ID NO: 9 - DNA, 21 bp, Artificial Sequence
  siRNA target human PIK3IP1, #1
agaggctaacctggaaactaa

SEQ ID NO: 10 - DNA, 21 bp, Artificial Sequence
  siRNA target human PIK3IP1, #2
tacactgttattcatggttaa

SEQ ID NO: 11 - DNA, 21 bp, Artificial Sequence
  PI5P4K alpha forward primer
aagaagaagcacttcgtagcg

SEQ ID NO: 12 - DNA, 22 bp, Artificial Sequence
  PI5P4K alpha reverse primer
atggctcagttcattgatcgag

SEQ ID NO: 13 - DNA, 21 bp, Artificial Sequence
  PI5P4K beta forward primer
ccacacgatcaatgagctgag

SEQ ID NO: 14 - DNA, 22 bp, Artificial Sequence
  PI5P4K beta reverse primer
tccttaaacttaaagcggctgg

SEQ ID NO: 15 - DNA, 19 bp, Artificial Sequence
  PI5P4K gamma forward primer
ccgggaagccagcgataag

SEQ ID NO: 16 - DNA, 21 bp, Artificial Sequence
  PI5P4K gamma reverse primer
agctgcactagaaactccaca

SEQ ID NO: 17 - DNA, 22 bp, Artificial Sequence
  PIK3IP1 forward primer
gctaggaggaactaccactttg

SEQ ID NO: 18 - DNA, 21 bp, Artificial Sequence
  PIK3IP1 reverse primer
gatggacaaggagcactgtta

SEQ ID NO: 19 - DNA, 3833 bp, Homo sapiens
  CDS (249)..(1469)
gcgggctgagcgcggatccgcggcgggcgcaggatacgggccggggcgcgagccgagcgcagtctgccgggccgagcgggcggagcgagccgagtggggctgagcgcgccggcggcggcgggcggagcggagcgcggcgcgccggggccgccgccggggggatgcggctgcctccccgggccggggtgtagagagggcgggtccccggcctcgggagcacggcggtggaggggacataggaggcggccatggcgacccccggcaacctagggtcctctgtcctggcgagcaagaccaagaccaagaagaagcacttcgtagcgcagaaagtgaagctgtttcgggccagcgacccgctgctcagcgtcctcatgtggggggtaaaccactcgatcaatgaactgagccatgttcaaatccctgttatgttgatgccagatgacttcaaagcctattcaaaaataaaggtggacaatcacctttttaacaaagaaaacatgccgagccatttcaagtttaaggaatactgcccgatggtcttccgtaacctgcgggagaggtttggaattgatgatcaagatttccagaattccctgaccaggagcgcacccctccccaacgactcccaggcccgcagtggagctcgttttcacacttcctacgacaaaagatacatcatcaagactattaccagtgaagacgtggccgaaatgcacaacatcctgaagaaataccaccagtacatagtggaatgtcatgggatcacccttcttccccagttcttgggcatgtaccggcttaatgttgatggagttgaaatatatgtgatagttacaagaaatgtattcagccaccgtttgtctgtgtataggaaatacgacttaaagggctctacagtggctagagaagctagtgacaaagaaaaggccaaagaactgccaactctgaaagataatgatttcattaatgagggccaaaagatttatattgatgacaacaacaagaaggtcttcctggaaaaactaaaaaaggatgttgagtttctggcccagctgaagctcatggactacagtctgctggtgggaattcatgatgtggagagagccgaacaggaggaagtggagtgtgaggagaacgatggggaggaggagggcgagagcgatggcacccacccggtgggaacccccccagatagccccgggaatacactgaacagctcaccacccctggctcccggggagttcgatccgaacatcgacgtctatggaattaagtgccatgaaaactcgcctaggaaggaggtgtacttcatggcaattattgacatccttactcattatgatgcaaaaaagaaagctgcccatgctgcaaaaactgttaaacatggcgctggcgcggagatctccaccgtgaacccagaacagtattcaaagcgctttttggactttattggccacatcttgacgtaacctcctgcgcagcctcggacagacatgaacattggatggacagaggtggcttcggtgtaggaaaaatgaaaaccaaactcagtgaagtactcatcttgcaggaagcaaacctccttgtttacatcttcaggccaagatgactgatttgggggctactcgctttacagctacctgattttcccagcatcgttctagctatttctgactttgtgtatatgtgtgtgtgtgtgtgttgggggggggtgagtgtgtgcgcgcgtgtgcattttaaaagtcataaattaattaaaacagatccacttcggtcagtatgtgtcccaacaaagaccctttgattccagctatggccgaatgaatgagtgagtgagtgagtgagtgaatgaacacacgtgtgggggaggggagaaggaagtgcatgatgtcaggcaccgtgttggcatcacacaacaaactgtggatcagttttttttttttttttttttttttggagttgaaagatgtgagacagtattcagaataatgaagataataatgatgattattataataatgatgatgattccaaggaaaaaacctacagcgaatgttccatttctaccccgcacgcagacactctccctaacactgataacctgagcccccagcactggacggaagaatgctggcgtctccgtgtgtactggttcagggttctggccccagccttgtcaggaccccctggtgtccagagcccccacccctcccgcaacaagcagctgatgccccagtgattctctatacatttttcacctcggccaatatgtccaggaaaactgcttacttctcttttcttgcctggagccttcattgttcacccttacgttgcaatataggaattaatgctacaaaataaaagtaaagcttacctgaaaagtgcatagtttggggcaatggtatctacatctcccactgtgggaaaaccagcaaagcatcaaaactctcaattctcctgttaccaaatgcagatctgaattataagatgtttatgtttgaccattgtttcaacaatgggattttgttacgaattatccctttaactgaaaccctcagttttactgtttacattattaggaaaacagggatatcttttgaatctaaaaatttgatgtacagcatgtgatttttgaagtttacatgtaaagtcacagtataggtgaaataacgtttgtcatattttgagacgtatcctgcagccatgtttttacgtgagtgttttagtcaaagtacatggtagacagtctttcacaataaaaggaaaaggatttttttttcctccaaatgtacatttatcaacctaatgattgatttttttaaaaagagatttcgccccagtctggtttatgaaagttcattgccctaaactgtgctgattgtttttaatcaagttataaatttccaacctagatcatgtatctaccaactctcctgcattttccaaaaggcattgagcttaaatattagtcttgcttagagtaggttatccacttacatgctgcgctaaagccatgcctttgaaactccttgtttaaaacatgatatgatttttgtgggcagtttcagaaaagaaaacaaacaaacaaaaatcgaccctttaattattacttgcaactcaacagatctccctgccgtactgccttttccaggaactttacttcagggctgtccagattgcagctgtgccccgtgtatgtggatctagttcacagagtctttggaagccagcagtcgtgccctccgtatactgtccactcattttatgtagatttggtatcctcagcagccagtgttaacaccactgtcacgtagtgtacagattcatcttttatgtatttaaagtaatccatactatgatttggtttttccctgcaccattaattctggcatcagatcagtttttgtgttgtgaagttctactgtggtttgacccaagaccacaaccatgagaccctgaagtaaagataaggtacacatacattatttgagtaactgtttccttgggggccaatctgtgtatgcttttagaagtttacagaatgcttttatttttgtctataacaaacagtctgtcatttatttctgttgataaaccatttggacagagtgaggacgtttgccctgttatctcctagtgctaacaatacactccagtcatgagccgggctttacaaataaagcacttttgatgactcacaagatgaatccttttttcctctgtcccaattgtgtgtctctgttccaaacacattttaaatactcggtcctgacagtgtctttagctaatccttgaagaaatgaaagtggaattgaatctttttagtttctaga

SEQ ID NO: 20 - protein, 406 aa, Homo sapiens
MATPGNLGSSVLASKTKTKKKHFVAQKVKLFRASDPLLSVLMWGVNHSINELSHVQIPVMLMPDDFKAYSKIKVDNHLFNKENMPSHFKFKEYCPMVFRNLRERFGIDDQDFQNSLTRSAPLPNDSQARSGARFHTSYDKRYIIKTITSEDVAEMHNILKKYHQYIVECHGITLLPQFLGMYRLNVDGVEIYVIVTRNVFSHRLSVYRKYDLKGSTVAREASDKEKAKELPTLKDNDFINEGQKIYIDDNNKKVFLEKLKKDVEFLAQLKLMDYSLLVGIHDVERAEQEEVECEENDGEEEGESDGTHPVGTPPDSPGNTLNSSPPLAPGEFDPNIDVYGIKCHENSPRKEVYFMAIIDILTHYDAKKKAAHAAKTVKHGAGAEISTVNPEQYSKRFLDFIGHILT

SEQ ID NO: 21 - DNA, 3628 bp, Homo sapiens
  CDS (207)..(1250)
gcagctgctgcggggcggggagccggagtcagccctggacctccggctgctcgcccgcctcgaagcccccagccccgcggcgcgtgcccccagccctctgggccgcccaggcaccaccacttgcgtcccctttagatctatggcccggtgcgggtcgggggtcccgtgtgcatatcaatgaactgagccatgttcaaatccctgttatgttgatgccagatgacttcaaagcctattcaaaaataaaggtggacaatcacctttttaacaaagaaaacatgccgagccatttcaagtttaaggaatactgcccgatggtcttccgtaacctgcgggagaggtttggaattgatgatcaagatttccagaattccctgaccaggagcgcacccctccccaacgactcccaggcccgcagtggagctcgttttcacacttcctacgacaaaagatacatcatcaagactattaccagtgaagacgtggccgaaatgcacaacatcctgaagaaataccaccagtacatagtggaatgtcatgggatcacccttcttccccagttcttgggcatgtaccggcttaatgttgatggagttgaaatatatgtgatagttacaagaaatgtattcagccaccgtttgtctgtgtataggaaatacgacttaaagggctctacagtggctagagaagctagtgacaaagaaaaggccaaagaactgccaactctgaaagataatgatttcattaatgagggccaaaagatttatattgatgacaacaacaagaaggtcttcctggaaaaactaaaaaaggatgttgagtttctggcccagctgaagctcatggactacagtctgctggtgggaattcatgatgtggagagagccgaacaggaggaagtggagtgtgaggagaacgatggggaggaggagggcgagagcgatggcacccacccggtgggaacccccccagatagccccgggaatacactgaacagctcaccacccctggctcccggggagttcgatccgaacatcgacgtctatggaattaagtgccatgaaaactcgcctaggaaggaggtgtacttcatggcaattattgacatccttactcattatgatgcaaaaaagaaagctgcccatgctgcaaaaactgttaaacatggcgctggcgcggagatctccaccgtgaacccagaacagtattcaaagcgctttttggactttattggccacatcttgacgtaacctcctgcgcagcctcggacagacatgaacattggatggacagaggtggcttcggtgtaggaaaaatgaaaaccaaactcagtgaagtactcatcttgcaggaagcaaacctccttgtttacatcttcaggccaagatgactgatttgggggctactcgctttacagctacctgattttcccagcatcgttctagctatttctgactttgtgtatatgtgtgtgtgtgtgtgttgggggggggtgagtgtgtgcgcgcgtgtgcattttaaaagtcataaattaattaaaacagatccacttcggtcagtatgtgtcccaacaaagaccctttgattccagctatggccgaatgaatgagtgagtgagtgagtgagtgaatgaacacacgtgtgggggaggggagaaggaagtgcatgatgtcaggcaccgtgttggcatcacacaacaaactgtggatcagttttttttttttttttttttttttggagttgaaagatgtgagacagtattcagaataatgaagataataatgatgattattataataatgatgatgattccaaggaaaaaacctacagcgaatgttccatttctaccccgcacgcagacactctccctaacactgataacctgagcccccagcactggacggaagaatgctggcgtctccgtgtgtactggttcagggttctggccccagccttgtcaggaccccctggtgtccagagcccccacccctcccgcaacaagcagctgatgccccagtgattctctatacatttttcacctcggccaatatgtccaggaaaactgcttacttctcttttcttgcctggagccttcattgttcacccttacgttgcaatataggaattaatgctacaaaataaaagtaaagcttacctgaaaagtgcatagtttggggcaatggtatctacatctcccactgtgggaaaaccagcaaagcatcaaaactctcaattctcctgttaccaaatgcagatctgaattataagatgtttatgtttgaccattgtttcaacaatgggattttgttacgaattatccctttaactgaaaccctcagttttactgtttacattattaggaaaacagggatatcttttgaatctaaaaatttgatgtacagcatgtgatttttgaagtttacatgtaaagtcacagtataggtgaaataacgtttgtcatattttgagacgtatcctgcagccatgtttttacgtgagtgttttagtcaaagtacatggtagacagtctttcacaataaaaggaaaaggatttttttttcctccaaatgtacatttatcaacctaatgattgatttttttaaaaagagatttcgccccagtctggtttatgaaagttcattgccctaaactgtgctgattgtttttaatcaagttataaatttccaacctagatcatgtatctaccaactctcctgcattttccaaaaggcattgagcttaaatattagtcttgcttagagtaggttatccacttacatgctgcgctaaagccatgcctttgaaactccttgtttaaaacatgatatgatttttgtgggcagtttcagaaaagaaaacaaacaaacaaaaatcgaccctttaattattacttgcaactcaacagatctccctgccgtactgccttttccaggaactttacttcagggctgtccagattgcagctgtgccccgtgtatgtggatctagttcacagagtctttggaagccagcagtcgtgccctccgtatactgtccactcattttatgtagatttggtatcctcagcagccagtgttaacaccactgtcacgtagtgtacagattcatcttttatgtatttaaagtaatccatactatgatttggtttttccctgcaccattaattctggcatcagatcagtttttgtgttgtgaagttctactgtggtttgacccaagaccacaaccatgagaccctgaagtaaagataaggtacacatacattatttgagtaactgtttccttgggggccaatctgtgtatgcttttagaagtttacagaatgcttttatttttgtctataacaaacagtctgtcatttatttctgttgataaaccatttggacagagtgaggacgtttgccctgttatctcctagtgctaacaatacactccagtcatgagccgggctttacaaataaagcacttttgatgactcacaagatgaatccttttttcctctgtcccaattgtgtgtctctgttccaaacacattttaaatactcggtcctgacagtgtctttagctaatccttgaagaaatgaaagtggaattgaatctttttagtttctagaaaaaaaaaaaaaaa

SEQ ID NO: 22 - protein, 347 aa, Homo sapiens
MLMPDDFKAYSKIKVDNHLFNKENMPSHFKFKEYCPMVFRNLRERFGIDDQDFQNSLTRSAPLPNDSQARSGARFHTSYDKRYIIKTITSEDVAEMHNILKKYHQYIVECHGITLLPQFLGMYRLNVDGVEIYVIVTRNVFSHRLSVYRKYDLKGSTVAREASDKEKAKELPTLKDNDFINEGQKIYIDDNNKKVFLEKLKKDVEFLAQLKLMDYSLLVGIHDVERAEQEEVECEENDGEEEGESDGTHPVGTPPDSPGNTLNSSPPLAPGEFDPNIDVYGIKCHENSPRKEVYFMAIIDILTHYDAKKKAAHAAKTVKHGAGAEISTVNPEQYSKRFLDFIGHILT

SEQ ID NO: 23 - DNA, 5732 bp, Homo sapiens
  CDS (482)..(1732)
ttgcgggaaagagccaaaccctggcgttggggggcccgggcggggagcccctcccgcggtccacagcgacgcctgcccagccctcctccccttccggctccggcacggggccccgaggcgttcggaggccaggcgggtttctgtcaggcccggggaggaggggcgggcggggcggccgctgcctccccgggacgggccgtaccacgcggacggggaggacggggccaggggactgcagggcggctgcaccgcccgggggcggggtgcggagcgggccggcgggctccccggggcggggcgggagggcggggcgtggggcggacggaaccaccggggcggggtgggaggtaacgggacgggcgcgaccatggcgcggtgagggagcgggggtggggatcggtccgggggaggcctgaggccgctggcttgtgcgctgtctccgccgcccccctctttcgccgccgccgccgccgccccgggcatgtcgtccaactgcaccagcaccacggcggtggcggtggcgccgctcagcgccagcaagaccaagaccaagaagaagcatttcgtgtgccagaaagtgaagctattccgggccagcgagccgatcctcagcgtcctgatgtggggggtgaaccacacgatcaatgagctgagcaatgttcctgttcctgtcatgctaatgccagatgacttcaaagcctacagcaagatcaaggtggacaatcatctcttcaataaggagaacctgcccagccgctttaagtttaaggagtattgccccatggtgttccgaaaccttcgggagaggtttggaattgatgatcaggattaccagaattcagtgacgcgcagcgcccccatcaacagtgacagccagggtcggtgtggcacgcgtttcctcaccacctacgaccggcgctttgtcatcaagactgtgtccagcgaggacgtggcggagatgcacaacatcttaaagaaataccaccagtttatagtggagtgtcatggcaacacgcttttgccacagttcctgggcatgtaccgcctgaccgtggatggtgtggaaacctacatggtggttaccaggaacgtgttcagccatcggctcactgtgcatcgcaagtatgacctcaagggttctacggttgccagagaagcgagcgacaaggagaaggccaaggacttgccaacattcaaagacaatgacttcctcaatgaagggcagaagctgcatgtgggagaggagagtaaaaagaacttcctggagaaactgaagcgggacgttgagttcttggcacagctgaagatcatggactacagcctgctggtgggcatccacgacgtggaccgggcagagcaggaggagatggaggtggaggagcgggcagaggacgaggagtgtgagaatgatggggtgggtggcaacctactctgctcctatggcacacctccggacagccctggcaacctcctcagctttcctcggttctttggtcctggggaattcgacccctctgttgacgtctatgccatgaaaagccatgaaagttcccccaagaaggaggtgtatttcatggccatcattgatatcctcacgccatacgatacaaagaagaaagctgcacatgctgccaaaacggtgaaacacggggcaggggccgagatctcgactgtgaaccctgagcagtactccaaacgcttcaacgagtttatgtccaacatcctgacgtagttctcttctaccttcagccagagccagagagctggatatggggtcggggatcgggagttagggagaagggtgtatttgggctagatgggagggtgggagcagagtcgggtttgggagggctttagcaatgagactgcagcctgtgacaccgaaagagactttagctgaagaggagggggatgtgctgtgtgtgcacctgctcacaggatgtaaccccaccttctgcttacccttgattttttctccccatttgacacccaggttaaaaaggggttccctttttggtaccttgtaaccttttaagataccttggggctagagatgacttcgtgggtttatttgggttttgtttctgaaatttcattgctccaggtttgctatttataatcatatttcatcagcctacccaccctccccatctttgctgagctctcagttcccttcaattaaagagatacccggtagacccagcacaagggtccttccagaaccaagtgctatggatgccagattggagaggtcagacacctcgccctgctgcatttgctcttgtctggattaactttgtaatttatggagtattgtgcacaacttcctccacctttcccttggattcaagtgaaaactgttgcattattcctccatcctgtctggaatacaccaggtcaacaccagagatctcagatcagaatcagagatctcagaggggaataagttcatcctcatgggatggtgaggggcaggaaagcggctgggctcttggacacctggttctcagagaaccctgtgatgatcacccaagccccaggctgtcttagcccctggagttcagaagtcctctctgtaaagcctgcctcccactaggtcaagaggaactagagtacctttggatttatcaggaccctcatgtttaaatggttatttccctttgggaaaacttcagaaactgatgtatcaaatgaggccctgtgccctcgatctatttccttcttccttctgacctcctcccaggcactcttacttctagccgaactcttagctctgggcagatctccaagcgcctggagtgctttttagcagagacacctcgttaagctccgggatgaccttgtaggagatctgtctccctgtgcctggagagttacagccagcaaggtgcccccatcttagagtgtggtgtccaaacgtgaggtggcttcctagttacatgaggatgtgatccaggaaatccagtttggaggcttgatgtgggttttgacctggcctcagccttggggctgtttttccttgttgccccgctctagacttttagcagatctgcagcccacaggcttttttggaaggagtggcttcctgcaggtgttccacctgccttcggagcctgccacccaggccctcagaactgagccacaggctgctctggccaggagagaaacagctctgttgttctgcattgggggaggtacattcctgcatcttctcaccccctcaaccaggaactggggatttgggatgagatatggtcagacttgtagataaccccaaagatgtgaagatcgcttgtgaaaccattttgaatgaatagattggtttcctgtggctccctccaaacctggccaagcccagcttccgaagcaggaaccagcactgtctctgtgcctgactcacagcatataggtcaggaaagaatggagacggcattcttggacttcactggggctgctggattggatgggaaaccttctggaagaggcagatgggggtcaaaccactgccttggccccaggaaggggccataggtaggtctgaacaactgccgcaagaccactacatgacttagggaacttgaaaccaactggctcatggagaaaacaaatttgacttgggaaagggattatgtaggaataatgtttggacttgatttccccacgtcataatgaagaatggaagtttggatctgctcctcgtcaggcgcagcatctctgaagcttggaaagctgtcttccagcagcctccgtggcctcgggttcctaccggcttctctgcatttggtctgctgatcatgttgccataatgtgtatggaaagtgtaacacattcttactggttaaagacgactaccaggtatctaacttgtttaacattgagtttgtgtgtgtgtgtgtatgtttgtgtgttttgtatattgtttacattttgagaggtagcattctgtttcaaatgctttttgtttttctgacagtattgttgactgggtcataacattttgagctgtggtttggtggattttcaatttttttttttaaaggtcattcgctgtgctatcttcaaaaccttgagtttggcccccaatttttggcattcaaatgtttaaaagctatttatcttggtttatacaagtttcctttctcttctttttgtcatggtattctatttggtctgcagtttgaatgtagagaaagtggactgatcccccaagcgttgtctgcccccactctttcctccttgggtcccgccattcttttactgggcagtcgagggcattggaggggaagtgactgccctcagcctcactccctggggccatgaagaaaagctaaacagtctcatggcatctcagaataatgttgggtctcccaagaagaaaggtgtaagaataacgacatggctgattaggcgaggccaggatagggctaaggccaggattcctggctggcatccagtcaccccttctcccatccttccccctcttcttccacaagtccgcagccgagacactgtagtctcccagccacagtgatgagtgccctggagactccactgacctctagatgaaggcccctggccctggttcctgttaattaacctctgggtctttgagtcccccagcacaaacttctttcctgtaccctgcggcttggggtcacagggcatgccgggaagccacagctgaggggcgcagactgaagcagtgctccacctctccttctttagctcaggggttgctggtctgtggcaggcgccacgagtggcccctgtggctgttctcagtggcagtctcttaagttcccaccacaggcagctctttatcccctctccctacttgactctttctcttgcctgtgcttttggcctcaaacaggcctgctggtagcgctcagggcgtgaggctacactcctgccctgcctttcctgtcttcatggtctgccagggcataccttggggaggtggaccaaagacccaggactttttgcagtagccagtcctaccccccagttgtctttttaccaattcagggtgggagagaaaactgcagcaccccagcatgtgagttactcaggtgttgggggctagaagggacagtgcgtttaaacaacactcagagctctggccttaaacctgtggccccccaagtctaggagcctcatctcttcctggcagtcatgcgggcaggaggtcctgaaagggaaaacccattcagacaactgttccccaatctaccagccatctgcaggggtcagtgaccgtggccctctccctcctctagaatgtgccacttatgaagagtgccccatggggaaaaggagactcagctgtcccttggcagcttgtgccagtatcccagggcagaagtttccacaggagcctcttgcccttgcgcagagccactgtgagaggcggtgggagccaacacccttgggggagggggcagtactgctcggcacatcccagcatcaggtcagatcattgaaattaaaaaatgtgaattaagttcatatccaccttttggggaagcaggacaaaccaccaccccaccaagtgtgtgacttctccatatcccactgcagtttccattttttaaatgggaattttcaatcccctgtgcttgtctaacgtctgctttaaaaagtttgagaccctgttactgtttgaaaatgcatgcatgttacgatgaatctccaacctgaggaaaaaaataaaactcaaaaagctttgtgta

SEQ ID NO: 24 - protein, 416 aa, Homo sapiens
MSSNCTSTTAVAVAPLSASKTKTKKKHFVCQKVKLFRASEPILSVLMWGVNHTINELSNVPVPVMLMPDDFKAYSKIKVDNHLFNKENLPSRFKFKEYCPMVFRNLRERFGIDDQDYQNSVTRSAPINSDSQGRCGTRFLTTYDRRFVIKTVSSEDVAEMHNILKKYHQFIVECHGNTLLPQFLGMYRLTVDGVETYMVVTRNVFSHRLTVHRKYDLKGSTVAREASDKEKAKDLPTFKDNDFLNEGQKLHVGEESKKNFLEKLKRDVEFLAQLKIMDYSLLVGIHDVDRAEQEEMEVEERAEDEECENDGVGGNLLCSYGTPPDSPGNLLSFPRFFGPGEFDPSVDVYAMKSHESSPKKEVYFMAIIDILTPYDTKKKAAHAAKTVKHGAGAEISTVNPEQYSKRFNEFMSNILT

SEQ ID NO: 25 - DNA, 3229 bp, Homo sapiens
  CDS (132)..(1397)
tcgccctgttcgcgcgtccgctgtccggcctccggtcacgtgacagcagcgcaggtgagcgccgcttccggggtcgggcgcctggatagctgccggctccggcttccacttggtcggttgcgcgggagactatggcgtcctcctcggtcccaccagccacggtatcggcggcgacagcaggccccggcccaggtttcggcttcgcctccaagaccaagaagaagcatttcgtgcagcagaaggtgaaggtgttccgggcggccgacccgctggtgggtgtgttcctgtggggcgtagcccactcgatcaatgagctcagccaggtgcctcccccggtgatgctgctgccagatgactttaaggccagctccaagatcaaggtcaacaatcaccttttccacagggaaaatctgcccagtcatttcaagttcaaggagtattgtccccaggtcttcaggaacctccgtgatcgatttggcattgatgaccaagattacttggtgtcccttacccgaaacccccccagcgaaagtgaaggcagtgatggtcgcttccttatctcctacgatcggactctggtcatcaaagaagtatccagtgaggacattgctgacatgcatagcaacctctccaactatcaccagtacattgtgaagtgccatggcaacacgcttctgccccagttcctggggatgtaccgagtcagtgtggacaacgaagacagctacatgcttgtgatgcgcaatatgtttagccaccgtcttcctgtgcacaggaagtatgacctcaagggttccctagtgtcccgggaagccagcgataaggaaaaggttaaagaattgcccacccttaaggatatggactttctcaacaagaaccagaaagtatatattggtgaagaggagaagaaaatatttctggagaagctgaagagagatgtggagtttctagtgcagctgaagatcatggactacagccttctgctaggcatccacgacatcattcggggctctgaaccagaggaggaagcgcccgtgcgggaggatgagtcagaggtggatggggactgcagcctgactggacctcctgctctggtgggctcctatggcacctccccagagggtatcggaggctacatccattcccatcggcccctgggcccaggagagtttgagtccttcattgatgtctatgccatccggagtgctgaaggagccccccagaaggaggtctacttcatgggcctcattgatatccttacacagtatgatgccaagaagaaagcagctcatgcagccaaaactgtcaagcatggggctggggcagagatctctactgtccatccggagcagtatgctaagcgattcctggattttattaccaacatctttgcctaagagactgcctggttctctctgatgttcaaggtggtggggttctgagacacttgggggaattgtggggatattctagccaccagttctcttcttcctttgctaaattcaggctgcaggctccttccatccagataactccatcctgtcgagtaggctctttctgaccctcagaaatacattgtcctttttcctctttgcccatttttcttccctctcttcctccccatgagaagtctgcttgtagtattagaatgttattgttgactctctcccaagtgccttgatctttgtaatatctcctgttgtttctatgatataggagctaggggaagggggttgtttgccttcttcaggacctgactggacagatggacctggctcaagcaactactctggatgcactttgctgtgtgggatgaactaaaagtgtctgaattttgctgataactttataaaactcactatggcatgcttccctcctggtgggccctaggatggatgacactcaagatactacagatgtgggtgcaggcatgcacacacacgatggaatatggccattcctacacaggtggggtagagagtgggtcagcagcctggcacctcacagaggtgggacctaagaggactcatgattatgcagagaattggattgggtctctgtcatagattgagtaatctcttcccttacctcaattccatctccacccatctctacatctgggcacagcaacccagagatggccaaaagcattcaagcctgggggaagatgtttgactattgctgctcttcaccagaacctcacacctctcctgggactggaacccttcagtgggtgtgtggccagttttggaggctggaatgatgggccagggtgtaggattcattctccatgtaaagtttcctttcatcctgcctagccatccccaaggtttatttccagaagaaaggaatatctctacttggatcaattctggtcatttcaagaggatggaggcctcaagtgtgggaacttcccctactccctggatgtgtgtacctagcacacttccttctcccacccctttttccagttggatttgtttttctgttctcttctgtcctgtcttatactgcaactgtgtctcctaggggacagatggccttctttgtcatcttcactctccacccccagagaggagtcagagccataactcaatcactcagcccctccaaagatagttgatgtgtgataatctcataatgttgagaaccctgatgagatacattgtcttcctctccctacaatgcctctggggccaaggcacccattcttcttgctatcctccatcccccttgaggcttccactttttttttttttagacataaagctgggcatcagcaactggcctgtggtgatgcaaagctgctttgctctgtatctggctggactgatctgtctcacaagaagccatgaggccatagggagaagctccctctccccttcatcttctgctccaaaggtggtagcaagaggagtacccagttaggggttggagcccccatataacatcttcctgtcagaagactgatggatctttttcattccaaccatctccctttcccccgatgaatgcaataaaactctgtgacaccagcaaccattgctctttagaaatgggttttctgatcatatggctgatgtgttatgggcagtatggatgtcttcatttgttgcttctgtttttcatcttttttgttttattaataaaaatttatgtatttgctcctgttactataataatacagggaataaattattcaatccaaatttctgtacaaaaaaaaaaa

SEQ ID NO: 26 - protein, 421 aa, Homo sapiens
MASSSVPPATVSAATAGPGPGFGFASKTKKKHFVQQKVKVFRAADPLVGVFLWGVAHSINELSQVPPPVMLLPDDFKASSKIKVNNHLFHRENLPSHFKFKEYCPQVFRNLRDRFGIDDQDYLVSLTRNPPSESEGSDGRFLISYDRTLVIKEVSSEDIADMHSNLSNYHQYIVKCHGNTLLPQFLGMYRVSVDNEDSYMLVMRNMFSHRLPVHRKYDLKGSLVSREASDKEKVKELPTLKDMDFLNKNQKVYIGEEEKKIFLEKLKRDVEFLVQLKIMDYSLLLGIHDIIRGSEPEEEAPVREDESEVDGDCSLTGPPALVGSYGTSPEGIGGYIHSHRPLGPGEFESFIDVYAIRSAEGAPQKEVYFMGLIDILTQYDAKKKAAHAAKTVKHGAGAEISTVHPEQYAKRFLDFITNIFA

SEQ ID NO: 27 - DNA, 2940 bp, Homo sapiens
  CDS (132)..(1397)
tcgccctgttcgcgcgtccgctgtccggcctccggtcacgtgacagcagcgcaggtgagcgccgcttccggggtcgggcgcctggatagctgccggctccggcttccacttggtcggttgcgcgggagactatggcgtcctcctcggtcccaccagccacggtatcggcggcgacagcaggccccggcccaggtttcggcttcgcctccaagaccaagaagaagcatttcgtgcagcagaaggtgaaggtgttccgggcggccgacccgctggtgggtgtgttcctgtggggcgtagcccactcgatcaatgagctcagccaggtgcctcccccggtgatgctgctgccagatgactttaaggccagctccaagatcaaggtcaacaatcaccttttccacagggaaaatctgcccagtcatttcaagttcaaggagtattgtccccaggtcttcaggaacctccgtgatcgatttggcattgatgaccaagattacttggtgtcccttacccgaaacccccccagcgaaagtgaaggcagtgatggtcgcttccttatctcctacgatcggactctggtcatcaaagaagtatccagtgaggacattgctgacatgcatagcaacctctccaactatcaccagtacattgtgaagtgccatggcaacacgcttctgccccagttcctggggatgtaccgagtcagtgtggacaacgaagacagctacatgcttgtgatgcgcaatatgtttagccaccgtcttcctgtgcacaggaagtatgacctcaagggttccctagtgtcccgggaagccagcgataaggaaaaggttaaagaattgcccacccttaaggatatggactttctcaacaagaaccagaaagtatatattggtgaagaggagaagaaaatatttctggagaagctgaagagagatgtggagtttctagtgcagctgaagatcatggactacagccttctgctaggcatccacgacatcattcggggctctgaaccagaggaggaagcgcccgtgcgggaggatgagtcagaggtggatggggactgcagcctgactggacctcctgctctggtgggctcctatggcacctccccagagggtatcggaggctacatccattcccatcggcccctgggcccaggagagtttgagtccttcattgatgtctatgccatccggagtgctgaaggagccccccagaaggaggtctacttcatgggcctcattgatatccttacacagtatgatgccaagaagaaagcagctcatgcagccaaaactgtcaagcatggggctggggcagagatctctactgtccatccggagcagtatgctaagcgattcctggattttattaccaacatctttgcctaagagactgcctggttctctctgatgttcaaggagctaggggaagggggttgtttgccttcttcaggacctgactggacagatggacctggctcaagcaactactctggatgcactttgctgtgtgggatgaactaaaagtgtctgaattttgctgataactttataaaactcactatggcatgcttccctcctggtgggccctaggatggatgacactcaagatactacagatgtgggtgcaggcatgcacacacacgatggaatatggccattcctacacaggtggggtagagagtgggtcagcagcctggcacctcacagaggtgggacctaagaggactcatgattatgcagagaattggattgggtctctgtcatagattgagtaatctcttcccttacctcaattccatctccacccatctctacatctgggcacagcaacccagagatggccaaaagcattcaagcctgggggaagatgtttgactattgctgctcttcaccagaacctcacacctctcctgggactggaacccttcagtgggtgtgtggccagttttggaggctggaatgatgggccagggtgtaggattcattctccatgtaaagtttcctttcatcctgcctagccatccccaaggtttatttccagaagaaaggaatatctctacttggatcaattctggtcatttcaagaggatggaggcctcaagtgtgggaacttcccctactccctggatgtgtgtacctagcacacttccttctcccacccctttttccagttggatttgtttttctgttctcttctgtcctgtcttatactgcaactgtgtctcctaggggacagatggccttctttgtcatcttcactctccacccccagagaggagtcagagccataactcaatcactcagcccctccaaagatagttgatgtgtgataatctcataatgttgagaaccctgatgagatacattgtcttcctctccctacaatgcctctggggccaaggcacccattcttcttgctatcctccatcccccttgaggcttccactttttttttttttagacataaagctgggcatcagcaactggcctgtggtgatgcaaagctgctttgctctgtatctggctggactgatctgtctcacaagaagccatgaggccatagggagaagctccctctccccttcatcttctgctccaaaggtggtagcaagaggagtacccagttaggggttggagcccccatataacatcttcctgtcagaagactgatggatctttttcattccaaccatctccctttcccccgatgaatgcaataaaactctgtgacaccagcaaccattgctctttagaaatgggttttctgatcatatggctgatgtgttatgggcagtatggatgtcttcatttgttgcttctgtttttcatcttttttgttttattaataaaaatttatgtatttgctcctgttactataataatacagggaataaattattcaatccaaatttctgtacaaaaaaaaaaa

SEQ ID NO: 28 - protein, 421 aa, Homo sapiens; identical to SEQ ID NO: 26

SEQ ID NO: 29 - DNA, 3175 bp, Homo sapiens
  CDS (132)..(1343)
tcgccctgttcgcgcgtccgctgtccggcctccggtcacgtgacagcagcgcaggtgagcgccgcttccggggtcgggcgcctggatagctgccggctccggcttccacttggtcggttgcgcgggagactatggcgtcctcctcggtcccaccagccacggtatcggcggcgacagcaggccccggcccaggtttcggcttcgcctccaagaccaagaagaagcatttcgtgcagcagaaggtgaaggtgttccgggcggccgacccgctggtgggtgtgttcctgtggggcgtagcccactcgatcaatgagctcagccaggtgcctcccccggtgatgctgctgccagatgactttaaggccagctccaagatcaaggagtattgtccccaggtcttcaggaacctccgtgatcgatttggcattgatgaccaagattacttggtgtcccttacccgaaacccccccagcgaaagtgaaggcagtgatggtcgcttccttatctcctacgatcggactctggtcatcaaagaagtatccagtgaggacattgctgacatgcatagcaacctctccaactatcaccagtacattgtgaagtgccatggcaacacgcttctgccccagttcctggggatgtaccgagtcagtgtggacaacgaagacagctacatgcttgtgatgcgcaatatgtttagccaccgtcttcctgtgcacaggaagtatgacctcaagggttccctagtgtcccgggaagccagcgataaggaaaaggttaaagaattgcccacccttaaggatatggactttctcaacaagaaccagaaagtatatattggtgaagaggagaagaaaatatttctggagaagctgaagagagatgtggagtttctagtgcagctgaagatcatggactacagccttctgctaggcatccacgacatcattcggggctctgaaccagaggaggaagcgcccgtgcgggaggatgagtcagaggtggatggggactgcagcctgactggacctcctgctctggtgggctcctatggcacctccccagagggtatcggaggctacatccattcccatcggcccctgggcccaggagagtttgagtccttcattgatgtctatgccatccggagtgctgaaggagccccccagaaggaggtctacttcatgggcctcattgatatccttacacagtatgatgccaagaagaaagcagctcatgcagccaaaactgtcaagcatggggctggggcagagatctctactgtccatccggagcagtatgctaagcgattcctggattttattaccaacatctttgcctaagagactgcctggttctctctgatgttcaaggtggtggggttctgagacacttgggggaattgtggggatattctagccaccagttctcttcttcctttgctaaattcaggctgcaggctccttccatccagataactccatcctgtcgagtaggctctttctgaccctcagaaatacattgtcctttttcctctttgcccatttttcttccctctcttcctccccatgagaagtctgcttgtagtattagaatgttattgttgactctctcccaagtgccttgatctttgtaatatctcctgttgtttctatgatataggagctaggggaagggggttgtttgccttcttcaggacctgactggacagatggacctggctcaagcaactactctggatgcactttgctgtgtgggatgaactaaaagtgtctgaattttgctgataactttataaaactcactatggcatgcttccctcctggtgggccctaggatggatgacactcaagatactacagatgtgggtgcaggcatgcacacacacgatggaatatggccattcctacacaggtggggtagagagtgggtcagcagcctggcacctcacagaggtgggacctaagaggactcatgattatgcagagaattggattgggtctctgtcatagattgagtaatctcttcccttacctcaattccatctccacccatctctacatctgggcacagcaacccagagatggccaaaagcattcaagcctgggggaagatgtttgactattgctgctcttcaccagaacctcacacctctcctgggactggaacccttcagtgggtgtgtggccagttttggaggctggaatgatgggccagggtgtaggattcattctccatgtaaagtttcctttcatcctgcctagccatccccaaggtttatttccagaagaaaggaatatctctacttggatcaattctggtcatttcaagaggatggaggcctcaagtgtgggaacttcccctactccctggatgtgtgtacctagcacacttccttctcccacccctttttccagttggatttgtttttctgttctcttctgtcctgtcttatactgcaactgtgtctcctaggggacagatggccttctttgtcatcttcactctccacccccagagaggagtcagagccataactcaatcactcagcccctccaaagatagttgatgtgtgataatctcataatgttgagaaccctgatgagatacattgtcttcctctccctacaatgcctctggggccaaggcacccattcttcttgctatcctccatcccccttgaggcttccactttttttttttttagacataaagctgggcatcagcaactggcctgtggtgatgcaaagctgctttgctctgtatctggctggactgatctgtctcacaagaagccatgaggccatagggagaagctccctctccccttcatcttctgctccaaaggtggtagcaagaggagtacccagttaggggttggagcccccatataacatcttcctgtcagaagactgatggatctttttcattccaaccatctccctttcccccgatgaatgcaataaaactctgtgacaccagcaaccattgctctttagaaatgggttttctgatcatatggctgatgtgttatgggcagtatggatgtcttcatttgttgcttctgtttttcatcttttttgttttattaataaaaatttatgtatttgctcctgttactataataatacagggaataaattattcaatccaaatttctgtacaaaaaaaaaaa

SEQ ID NO: 30 - protein, 403 aa, Homo sapiens
MASSSVPPATVSAATAGPGPGFGFASKTKKKHFVQQKVKVFRAADPLVGVFLWGVAHSINELSQVPPPVMLLPDDFKASSKIKEYCPQVFRNLRDRFGIDDQDYLVSLTRNPPSESEGSDGRFLISYDRTLVIKEVSSEDIADMHSNLSNYHQYIVKCHGNTLLPQFLGMYRVSVDNEDSYMLVMRNMFSHRLPVHRKYDLKGSLVSREASDKEKVKELPTLKDMDFLNKNQKVYIGEEEKKIFLEKLKRDVEFLVQLKIMDYSLLLGIHDIIRGSEPEEEAPVREDESEVDGDCSLTGPPALVGSYGTSPEGIGGYIHSHRPLGPGEFESFIDVYAIRSAEGAPQKEVYFMGLIDILTQYDAKKKAAHAAKTVKHGAGAEISTVHPEQYAKRFLDFITNIFA

SEQ ID NO: 31 - DNA, 3085 bp, Homo sapiens
  CDS (132)..(1253)
tcgccctgttcgcgcgtccgctgtccggcctccggtcacgtgacagcagcgcaggtgagcgccgcttccggggtcgggcgcctggatagctgccggctccggcttccacttggtcggttgcgcgggagactatggcgtcctcctcggtcccaccagccacggtatcggcggcgacagcaggccccggcccaggtttcggcttcgcctccaagaccaagaagaagcatttcgtgcagcagaaggtgaaggtgttccgggcggccgacccgctggtgggtgtgttcctgtggggcgtagcccactcgatcaatgagctcagccaggtgcctcccccggtgatgctgctgccagatgactttaaggccagctccaagatcaaggtcaacaatcaccttttccacagggaaaatctgcccagtcatttcaagttcaaggagtattgtccccaggtcttcaggaacctccgtgatcgatttggcattgatgaccaagattacttgtacattgtgaagtgccatggcaacacgcttctgccccagttcctggggatgtaccgagtcagtgtggacaacgaagacagctacatgcttgtgatgcgcaatatgtttagccaccgtcttcctgtgcacaggaagtatgacctcaagggttccctagtgtcccgggaagccagcgataaggaaaaggttaaagaattgcccacccttaaggatatggactttctcaacaagaaccagaaagtatatattggtgaagaggagaagaaaatatttctggagaagctgaagagagatgtggagtttctagtgcagctgaagatcatggactacagccttctgctaggcatccacgacatcattcggggctctgaaccagaggaggaagcgcccgtgcgggaggatgagtcagaggtggatggggactgcagcctgactggacctcctgctctggtgggctcctatggcacctccccagagggtatcggaggctacatccattcccatcggcccctgggcccaggagagtttgagtccttcattgatgtctatgccatccggagtgctgaaggagccccccagaaggaggtctacttcatgggcctcattgatatccttacacagtatgatgccaagaagaaagcagctcatgcagccaaaactgtcaagcatggggctggggcagagatctctactgtccatccggagcagtatgctaagcgattcctggattttattaccaacatctttgcctaagagactgcctggttctctctgatgttcaaggtggtggggttctgagacacttgggggaattgtggggatattctagccaccagttctcttcttcctttgctaaattcaggctgcaggctccttccatccagataactccatcctgtcgagtaggctctttctgaccctcagaaatacattgtcctttttcctctttgcccatttttcttccctctcttcctccccatgagaagtctgcttgtagtattagaatgttattgttgactctctcccaagtgccttgatctttgtaatatctcctgttgtttctatgatataggagctaggggaagggggttgtttgccttcttcaggacctgactggacagatggacctggctcaagcaactactctggatgcactttgctgtgtgggatgaactaaaagtgtctgaattttgctgataactttataaaactcactatggcatgcttccctcctggtgggccctaggatggatgacactcaagatactacagatgtgggtgcaggcatgcacacacacgatggaatatggccattcctacacaggtggggtagagagtgggtcagcagcctggcacctcacagaggtgggacctaagaggactcatgattatgcagagaattggattgggtctctgtcatagattgagtaatctcttcccttacctcaattccatctccacccatctctacatctgggcacagcaacccagagatggccaaaagcattcaagcctgggggaagatgtttgactattgctgctcttcaccagaacctcacacctctcctgggactggaacccttcagtgggtgtgtggccagttttggaggctggaatgatgggccagggtgtaggattcattctccatgtaaagtttcctttcatcctgcctagccatccccaaggtttatttccagaagaaaggaatatctctacttggatcaattctggtcatttcaagaggatggaggcctcaagtgtgggaacttcccctactccctggatgtgtgtacctagcacacttccttctcccacccctttttccagttggatttgtttttctgttctcttctgtcctgtcttatactgcaactgtgtctcctaggggacagatggccttctttgtcatcttcactctccacccccagagaggagtcagagccataactcaatcactcagcccctccaaagatagttgatgtgtgataatctcataatgttgagaaccctgatgagatacattgtcttcctctccctacaatgcctctggggccaaggcacccattcttcttgctatcctccatcccccttgaggcttccactttttttttttttagacataaagctgggcatcagcaactggcctgtggtgatgcaaagctgctttgctctgtatctggctggactgatctgtctcacaagaagccatgaggccatagggagaagctccctctccccttcatcttctgctccaaaggtggtagcaagaggagtacccagttaggggttggagcccccatataacatcttcctgtcagaagactgatggatctttttcattccaaccatctccctttcccccgatgaatgcaataaaactctgtgacaccagcaaccattgctctttagaaatgggttttctgatcatatggctgatgtgttatgggcagtatggatgtcttcatttgttgcttctgtttttcatcttttttgttttattaataaaaatttatgtatttgctcctgttactataataatacagggaataaattattcaatccaaatttctgtacaaaaaaaaaaa

SEQ ID NO: 32 - protein, 373 aa, Homo sapiens
MASSSVPPATVSAATAGPGPGFGFASKTKKKHFVQQKVKVFRAADPLVGVFLWGVAHSINELSQVPPPVMLLPDDFKASSKIKVNNHLFHRENLPSHFKFKEYCPQVFRNLRDRFGIDDQDYLYIVKCHGNTLLPQFLGMYRVSVDNEDSYMLVMRNMFSHRLPVHRKYDLKGSLVSREASDKEKVKELPTLKDMDFLNKNQKVYIGEEEKKIFLEKLKRDVEFLVQLKIMDYSLLLGIHDIIRGSEPEEEAPVREDESEVDGDCSLTGPPALVGSYGTSPEGIGGYIHSHRPLGPGEFESFIDVYAIRSAEGAPQKEVYFMGLIDILTQYDAKKKAAHAAKTVKHGAGAEISTVHPEQYAKRFLDFITNIFA